(19) United States
(12) Patent Application Publication
(10) Pub. No.: US 2023/0141224 A1
(43) Pub. Date: May 11, 2023

(54) FIBROBLAST MEDIATED EXPANSION AND AUGMENTATION OF IMMUNE REGULATORY CELLS FOR TREATMENT OF ACUTE RESPIRATORY DISTRESS SYNDROME (ARDS)

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/906,883

(22) PCT Filed: Mar. 2, 2021
(86) PCT No.: PCT/US2021/020459

§ 371 Date: Sep. 21, 2022

Related U.S. Application Data
(60) Provisional application No. 63/017,068, filed on Apr. 29, 2020.
(60) Provisional application No. 63/003,731, filed on Apr. 1, 2020.

(51) Int. Cl. A61K 35/17; C12N 5/0783; A61K 35/33; A61K 35/51; A61P 29/00; A61P 11/00; A61K 45/06

(52) U.S. Cl. CPC A61K 35/17; C12N 5/0636; A61K 35/33; A61K 35/51; A61P 29/00; A61P 11/00; A61K 45/06; C12N 2502/1323

(57) ABSTRACT

Embodiments of the disclosure encompass methods and compositions related to treatment of Acute Respiratory Distress Syndrome caused by any reason, including caused by a coronavirus, for example. In particular embodiments, fibroblasts are delivered to an individual in need thereof to stimulate generation of T regulatory cells that may or may not be FoxP3-positive, and/or immune regulatory cells previously exposed to fibroblasts are delivered to the individual.

This application claims priority to U.S. Provisional Patent Application Ser. No. 63/003,731, filed Apr. 1, 2020, and also to U.S. Provisional Patent Application Ser. No. 63/017,068, filed Apr. 29, 2020, both of which applications are incorporated by reference herein in their entirety.

### TECHNICAL FIELD

Embodiments of the disclosure include at least the fields of cell biology, molecular biology, and medicine.

### BACKGROUND

COVID-19 presents a high mortality rate, estimated at 3.4% by the World Health Organization [1]. The rapid spread of the virus (estimated reproductive number R₀ 2.2-3.6 [2, 3] is causing a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died [4-8]. A common observation among patients with severe COVID-19 infection is an inflammatory response localized to the lower respiratory tract [9-11]. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death [8, 12]. Draconian measures have been put in place in an attempt to curtail the impact of the COVID-19 epidemic on population health and healthcare systems. However, WHO has now classified COVID-19 a pandemic [1]. At the present time, there is neither a vaccine nor specific antiviral treatments for seriously ill individuals infected with COVID-19. Crucially, no options are available for those individuals with rapidly progressing ARDS evolving to organ failure. Although supportive care is provided whenever possible, including mechanical ventilation and support of vital organ functions, it is insufficient in most severe cases. Therefore, there is an urgent need for novel therapies that can dampen the excessive inflammatory response in the lungs, associated with the immunopathological cytokine storm, and accelerate the regeneration of functional lung tissue in individuals with COVID-19.

More generally, acute respiratory distress syndrome (ARDS) is a sudden onset form of respiratory failure caused by a variety of factors. ARDS generally presents with progressive hypoxemia, dyspnea and increased work of breathing [1]. Patients often require mechanical ventilation and supplemental oxygen. Over the years, our understanding of ARDS has advanced significantly. However, ARDS is still associated with significant morbidity and mortality and therapeutic strategies to mitigate the foregoing have resulted in limited translational success. Part of this failure stems from heterogeneity associated with this disease.

ARDS can be caused by bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS. ARDS is often associated with fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Many patients who develop ARDS do not survive. The risk of death increases with age and severity of illness. Of the people who do survive ARDS, some recover completely while others experience lasting damage to their lungs.

There are currently no effective pharmacologic therapies for treatment or prevention of ARDS. While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel therapeutic cells and combinations useful in the treatment of ARDS and methods of treatment for ARDS and conditions related thereto through the administration of such novel therapeutic agents.

### BRIEF SUMMARY

The present disclosure is directed to systems, methods, compositions and kits that are for preventing or treating Acute Respiratory Distress Syndrome (ARDS) in an individual in need thereof. In specific cases, the ARDS is characterized by fluid build-up in the alveoli of the lungs. Symptoms include severe shortness of breath; labored and unusually rapid breathing; low blood pressure; and/or confusion and extreme tiredness. The underlying cause may be infection, including viral infection, sepsis, inhalation of harmful substances, severe pneumonia, and/or head, chest or other major injury. In specific cases, the individual has coronavirus, including is positive for COVID-19 (SARS-CoV-2). In preventative cases, the individual may or may not be asymptomatic; an individual may have been exposed to coronavirus and is or is not displaying one or more symptoms. In some embodiments, the ARDS is associated with at least one of the following: bacterial pneumonia, viral pneumonia, sepsis, head injury, chest injury, burns, blood transfusions, near drowning, aspiration of gastric contents, pancreatitis, intravenous drug use, abdominal trauma, or acute radiation syndrome.

In particular embodiments, the method comprises administering to the individual an effective amount of: (a) fibroblasts and/or fibroblast-derived products (including exosomes) at a concentration and frequency to allow for the fibroblasts to stimulate generation of T regulatory cells in vivo; and/or (b) immune regulatory cells, wherein the immune regulatory cells have been exposed ex vivo or in vitro under suitable conditions to fibroblasts and/or fibroblast-derived exosomes. The immune regulatory cells may be obtained by culture of lymphocytes with fibroblasts to produce T cells and/or B cells. In specific cases, the immune regulatory cells are T cells. The T regulatory cells may be generated in vivo from T cell progenitors, naïve T cells, Th1, Th2, Th3, Th9, Th17 T cells, or a mixture thereof. The T regulatory cells may express a marker selected from the group consisting of CD4, CD25, CD73, CD105, LAP, TGF-beta, CTLA-4, GITR ligand, neuropilin-1, CTLA-4, FoxP3, CD127, GARP, and a combination thereof. The T regulatory cells may or may not express FoxP3 and/or membrane-bound TGF-beta. The T regulatory cells may suppress ability of T cells to proliferate in response to one or more mitogens. The T regulatory cells may suppress ability of immature dendritic cells to mature into differentiated dendritic cells. Dendritic cell maturation may be associated with upregulation of expression of one or more markers selected from the group consisting of: HLA-II, CD40, CD80, CD86, and a combination thereof. Dendritic cell maturation may be associated with enhanced ability to activate proliferation of allogeneic T cells. Dendritic cell maturation may be associated with enhanced ability to induce production of interferon gamma from allogeneic T cells.

In specific embodiments, in (a) the fibroblasts are allogeneic, autologous or syngeneic with respect to the T regulatory cells. In specific embodiments, in (b) the immune regulatory cells are allogeneic, autologous or syngeneic with respect to the individual. The fibroblasts may substitute for immature dendritic cells in order to stimulate Treg generation in vivo. The fibroblasts may be administered with immature dendritic cells in order to stimulate Treg generation in vivo.

In certain embodiments, for the individual the ARDS is comprised of neutrophil infiltration into the alveolar space; complement activation in the lung; and/or enhanced expression of one or more inflammatory cytokines (such as selected from the group consisting of: IL-1, IL-6, IL-8, IL-11, IL-12, IL-18, IL-21, IL-17, IL-23, IL-27, IL33, TNF-alpha, HMGB-1, and a combination thereof).

In some embodiments, one or more NF-kappa B inhibitors are administered to the individual, such as a NF-kappa B inhibitor is selected from the group consisting of: Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, and a combination thereof.

In some embodiments, the method further comprises administration of one or more malaria drugs to the individual In some embodiments, the method further comprises administration of one or more adjuvants to the individual. The individual may be provided an effective amount of: (a) one or more peptides selected from the group consisting of: BPC-157, beta thymosine, Pam3CysSerLys4, functional derivatives thereof, and a mixture thereof; (b) one or more activators of one or more toll like receptors; (c) chloroquine, hydroxychloroquine, a functionally active derivative thereof, or a mixture thereof; (d) resveratrol and/or a functionally active derivative thereof; (e) losartan and/or a functionally active derivative thereof; (f) azithromycin and/or a functionally active derivative thereof; or (g) a combination thereof.

Functionally active derivatives of chloroquine or hydroxychloroquine include SKM13, SKM14, a metal-chloroquine, or a combination thereof. Functionally active derivatives of resveratrol include trans-resveratrol (3,5,4′-trihydroxystilbene); cis-resveratrol; Pterostilbene (3,5-Dimethoxy-4′ Hydroxystilbene); Trimethoxystilbene; Tetramethoxystilbene; Pentamethoxystilbene; Dihydroxystilbene; Tetrahydroxystilbene; Hexahydroxystilbene; 4′-Bromoresveratrol; 3,4,5-Trimethoxy-4′-bromo-trans-stilbene (BTS); 3,4,5-Trimethoxy-4′-bromo-cis-stilbene (BCS); 2-Chlororesveratrol; 4-Iodoresveratrol; or a combination thereof. A functionally active derivative of losartan is a Losartan Nitroderivative. Functionally active derivatives of azithromycin include 4″-O-(benzamido)alkyl carbamates of 11,12-cyclic carbonate AZM; 4″-O-(benzamido)butyl carbamates of 11,12-cyclic carbonate AZM; or combinations thereof.

In some embodiments, there is a method for generating a T cell population capable of suppressing pulmonary edema from any cause, wherein the T cell population comprises CD4+CD25+ regulatory T cells that are generated from freshly isolated CD4+CD25− T cells, the method comprising: isolating CD4+CD25− T cells from a sample comprising T cells obtained from a mammalian individual; contacting the isolated CD4+CD25− T cells in a culture vessel with one or more CD4+CD25+ induction agents for a time period sufficient to generate CD4+CD25+ regulatory T cells; and selecting the CD4+CD25+ cells. In specific cases, the induction agent is a population of fibroblasts and/or products derived therefrom. The population of fibroblast may be allogeneic, autologous, or xenogeneic to the T cell population. The CD4+CD25+ T cells may express FoxP3. In specific embodiments, the regulatory T cells are capable of in vitro cell-to-cell contact dependent suppression of the proliferation of freshly isolated CD4+CD25− responder T cells after re-exposure to a cognate antigen. The method may further comprise expanding the CD4+CD25+ antigen-specific regulatory T cell population. The method may further comprise administering a pharmaceutically acceptable composition comprising at least a portion of the expanded CD4+CD25+ antigen-specific regulatory T cell population to an individual in need thereof.

In some embodiments, methods for treating ARDS comprise administering to an individual an effective amount of antigen presenting cells in combination with fibroblasts. In some embodiments, methods for treating ARDS comprise administering to an individual an effective amount of antigen presenting cells in combination with mesenchymal stem cells (MSCs). In some embodiments, methods for treating ARDS comprise administering to an individual an effective amount of antigen presenting cells in combination with fibroblasts and MSC s. The antigen presenting cells may be immature antigen presenting cells. The antigen presenting cells may be monocytes and/or dendritic cells, including immature dendritic cells. The antigen presenting cell may decrease the mRNA and/or protein expression of one or more inflammatory cytokines in cells of the individual. The inflammatory cytokine(s) may be a cytokine selected from the group consisting of IL-6, IL1a, TNF-alpha, IL1 beta, Interferon gamma, IL-8, CXCL-1, CCL-2, HMGB-1, IL-11, IL-17, IL-18, IL-21, IL-22, IL-27, IL-33, TNF-beta, and a combination thereof. The antigen presenting cell may increase the mRNA and/or protein expression of one or more anti-inflammatory cytokines in cells of the individual. The anti-inflammatory cytokine(s) may be a cytokine selected from the group consisting of IL-10, TGF-beta, IL-4, TGS-6, galectin-1, galectin-3, galectin-9, and a combination thereof. In some embodiments, the dendritic cells stimulate the generation of T regulatory cells, including FoxP3⁺ T regulatory cells. The T regulatory cells may be capable of inhibiting T cells that have been stimulated through a T cell receptor and/or a costimulatory molecule.

The fibroblasts may be plastic adherent. The fibroblasts may be treated with a composition capable of activating NF-kappa B, such as hydrogen peroxide, ozone, TNF-alpha, interleukin-1, osmotic shock, mechanical agitation, or a combination thereof. The NF-kappa B activation may be transient. The NF-kappa B activation may endow the fibroblasts with an ability to produce IL-10, IL-35, IL-37, or a combination thereof. The fibroblasts may be derived from tissue selected from the group consisting of placenta, cord blood, mobilized peripheral blood, omentum, hair follicle, skin, bone marrow, adipose tissue, Wharton's Jelly, and a combination thereof. In some embodiments, peripheral blood mobilization refers to blood extracted from a patient who has received one or more treatments which promotes entrance of fibroblasts into circulation. The mobilization of fibroblasts and/or fibroblast progenitors into circulation is accomplished by administration of an agent selected from the group consisting of VLA-5 antibodies, G-CSF, M-CSF, GM-CSF, FLT-3L, TNF-alpha, EGF, FGF-1, FGF-2, FGF-5, VEGF, and a combination thereof.

The MSCs may express CD73, CD90, CD105, PD-L1, membrane-bound TGF-beta, indolamine 2,3 deoxygenase, or a combination thereof. The MSCs may be plastic-adherent. In some embodiments, the MSCs do not express HLA-II, CD14, and CD34. MSCs may be derived from one or more tissues, including for example, bone marrow, cord blood, placenta, umbilical cord, Wharton's jelly, and/or adipose tissue. In some embodiments, the MSCs are treated with an inflammatory stimuli at a sufficient concentration and for a sufficient time period to enhance anti-inflammatory properties of the MSCs. The inflammatory stimuli may be selected from the group consisting of interferon gamma, interleukin-1, interleukin-6, interleukin-8, TNF-alpha, interleukin-11, interleukin 12, interleukin-15, interleukin-17, interleukin-18, interleukin-33 and a combination thereof. The anti-inflammatory properties enhanced by the inflammatory stimuli may comprise an increased expression of IL-10 and/or TSG-6. The anti-inflammatory properties enhanced by the inflammatory stimuli may comprise an increased ability to inhibit production of TNF-alpha from endotoxin activity macrophages.

In some embodiments, T regulatory cells, type 2 macrophages, myeloid suppressor cells, hematopoietic stem cells (including hematopoietic stem cells that express CD34), or a combination thereof together with immature dendritic cells and with or without fibroblasts and/or MSCs are administered to the individual.

In some embodiments, the immature dendritic cell is derived from a monocyte precursor, including a monocyte and/or a type 2 monocyte. The monocyte precursor may be autologous or allogenic with respect to the individual. The monocyte precursor may be plastic adherent and/or express CD14. To derive immature dendritic cells from monocyte precursors, the monocyte precursors may be exposed to one or more agents capable of activating the GM-CSF receptor. In some embodiments, the monocyte precursor is exposed to the agent capable of activating GM-CSF for 1, 2, 3, 4, 5, 6, 7, 8, 9, 10, 11, 12, 13, 14, 15, 16, 17, 18, 19, 20, 21, 22, or 23 hours. In some embodiments, the monocyte precursor is exposed to the agent capable of activating GM-CSF for less than one hour. In some embodiments, the monocyte precursor is exposed to the agent capable of activating GM-CSF for 1, 2, 3, 4, 5, 6, 7, 8, 9, 10, 11, 12, 13, 14, or more days. In some embodiments, the agent capable of activating the GM-CSF receptor is GM-CSF.

In some embodiments, the immature dendritic cell is derived from a hematopoietic stem cell precursor. The hematopoietic stem cell precursor may be allogenic or autologous with respect to the individual. The hematopoietic stem cell precursor may or may not be plastic adherent. The hematopoietic stem cell precursor may express CD34 and/or CD133. To derive immature dendritic cells from hematopoietic stem cell precursors, the hematopoietic stem cell precursors may be exposed to one or more agents capable of activating the GM-CSF receptor. In some embodiments, the hematopoietic stem cell precursors is exposed to the agent capable of activating GM-CSF for 1, 2, 3, 4, 5, 6, 7, 8, 9, 10, 11, 12, 13, 14, 15, 16, 17, 18, 19, 20, 21, 22, or 23 hours. In some embodiments, the hematopoietic stem cell precursors is exposed to the agent capable of activating GM-CSF for less than one hour. In some embodiments, the hematopoietic stem cell precursors is exposed to the agent capable of activating GM-CSF for 1, 2, 3, 4, 5, 6, 7, 8, 9, 10, 11, 12, 13, 14, or more days. In some embodiments, the agent capable of activating the GM-CSF receptor is GM-CSF.

In some embodiments, a dendritic cell precursor is exposed to a combination of GM-CSF and IL-4 for 1, 2, 3, 4, 5, 6, 7, 8, 9, 10, 11, 12, 13, 14, or more days. In some embodiments, the immature dendritic cells is contacted with one or more inhibitors of NF-kappa B. The NF-kappa B inhibitor may be a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ isotonic), and a combination thereof.

In some embodiments, immature dendritic cells are exposed, in vitro or in vivo, to rapamycin to suppress maturation of the immature dendritic cells.

In some embodiments, the immature dendritic cells express CD11c and/or DEC-205. The immature dendritic cells may express higher levels of IL-10, PD-L1, and/or TSG-6 compared to the cell from which the immature dendritic cells were derived.

In some embodiments, the immature dendritic cells are modified to maintain the immature state. The immature dendritic cells may be modified by gene-editing techniques. Genes associated with dendritic cell maturation may be modified, mutated, silenced, and/or deleted. In some embodiments, genes associated with dendritic cell maturation are silenced by RNAi, technology, antisense oligonucleotides, ribozymes, or a combination thereof. Genes associated with dendritic cell maturation include, for example, NF-kappa B, IL-12, CD40, CD80, and CD86.

In some embodiments, the individual is administered an anti-viral composition in addition to one or more of the cells encompassed herein. The anti-viral composition may be selected from the group consisting of chloroquine, hydroxychloroquine, remdesivir, lopinavir, reproxalap, apabetalone, tradipitant, arbidol umifenovir, ganovo danoprevir, riavax tertomotide, thymosin alpha 1, ifenprodil (NP-120), avigan favipiravir, aviptadil, oseltamivir, and a combination thereof.

In some embodiments, fibroblast-derived products, such as exosomes (including exosomes derived from one or more of the cells disclosed herein), are administered to the individual alone or in combination with one or more of the cells and/or compositions disclosed herein. In some embodiments, the fibroblast-derived products, such as exosomes, are derived from dendritic cells, fibroblasts, monocytes, or a combination thereof. The fibroblast-derived products, such as exosomes, may express annexin V. The fibroblast-derived products, such as exosomes, may be concentrated by ultracentrifugation.

Certain methods encompassed herein for treating an individual, including an individual with ARDS, comprise the steps of obtaining tissue, dissociating the tissue to obtain a single cell suspension, isolating CD14+ cells from the single cell suspension, and exposing said CD14+ cells to GM-CSF and/or IL-4 at a concentration sufficient to generate immature dendritic cells. The CD14+ cells may be cultured before, after, and/or simultaneously with the exposure to GM-CSF and/or IL-4.

In some embodiments, the immature dendritic cells with fibroblasts and/or MSCs are administered in combination with one or more additional immune suppressive compositions to an individual. The immune suppressive composition may inhibit T cell proliferation, T cell production, antigen presenting cell function, T cell activity, and/or B cell activity. In some embodiments, the immune suppressive composition is selected from the group consisting of cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), Monoclonal antibodies, basiliximab (Simulect), daclizumab (Zinbryta), and a combination thereof.

The methods encompassed herein for treating an individual may inhibit a cytokine storm associated with any of disease encompassed herein. The cytokine storm may comprise an excessive production of inflammatory cytokines in any cell or tissue in the individual. The inflammatory cytokines may be associated with an increase in blood vessel permeability, an increase in pro-thrombotic molecules on the vasculature, a decrease in anti-thrombotic molecules on endothelial cells, an induction of hypotension, an induction of vascular leakage, or a combination thereof. The pro-thrombotic molecules may be tissue factor, von Willebrand factor, plasminogen activator inhibitor, or a combination thereof. The anti-thrombotic molecule may be nitric oxide synthase, thrombomodulin, protein C receptor, or a combination thereof. The inflammatory cytokines may be capable of inducing endothelial cell expression of genes selected from the group consisting of IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, Homo sapiens Down syndrome critical region, FI5613 containing ZNF gene family member, IL8, ELFR, Homo sapiens mRNA for dual specificity phosphatase MKP-5, Homo sapiens regulator of G protein signaling 10 mRNA complete, Homo sapiens Wnt-13 Mma, Homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 Homo sapiens mRNA, CDNA DKFZp434N2072,RPL26, and HR gene for hairless protein, regulator of G protein signaling. In some embodiments, the inflammatory cytokines comprise at least one of IL-1, IL-6, IL-12, IL-18, IL-33, TNF-alpha, IFN-gamma, HMGB-1, and IL-15.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWING

For a more complete understanding of the present disclosure, reference is now made to the following descriptions taken in conjunction with the accompanying drawing, in which:

FIG. 1 shows lung edema assessment quantified by the ratio of lung wet weight to body weight ratios (LWW/BW). From left to right, the bars represent control, lipopolysaccharides (LPS) and fibroblasts, lipopolysaccharides and T regulatory cells, and a combination of lipopolysaccharides, fibroblasts, and T regulatory cells.

### DETAILED DESCRIPTION

### I. EXAMPLES OF DEFINITIONS

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the individual. For example, one method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Tissue transplanted by such procedures is referred to as an allograft or allotransplant.

As used herein, the terms “allostimulatory” and “alloreactive” refer to stimulation and reaction of the immune system in response to an allologous antigens, or “alloantigens” or cells expressing a dissimilar HLA haplotype.

As used herein, “autologous” refers to tissues or cells that are derived or transferred from the same individual's body.

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” refers to any molecule having structural, regulatory or biochemical functions.

The term “cell culture” as used herein refers to an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂, although in other cases these are altered. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlioStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. PARTICULAR EMBODIMENTS

Particular embodiments of the disclosure concern fibroblast-mediated expansion and augmentation of immune regulatory cells for treatment of ARDS.

Disclosed are cell populations, culture conditions, compositions, systems, and therapeutic means for preventing, ameliorating, delay the onset of, reduce the severity of, or reversing ARDS through stimulation of immune regulatory cells in vivo using fibroblasts, or administration of immune regulatory cells that in an in vitro or ex vivo manner have been exposed under suitable conditions to fibroblasts. In one embodiment, immune regulatory cells are generated by culture or contact with fibroblasts. In some embodiments, in vitro generation of immune regulatory cells may be obtained by culture of lymphocytes with fibroblasts to generate T and/or B cells that suppress T or B cell responses. The immune regulatory cells are useful for treatment of ARDS.

A method of preventing or treating Acute Respiratory Distress Syndrome (ARDS) comprising administration to an individual in need thereof of an effective amount of fibroblasts at a concentration and frequency to allow for the fibroblasts to stimulate generation of T regulatory cells in vitro or in vivo from T cell progenitors, naïve T cells, Th1, Th2, Th3, Th9, or Th17 T cells.

Methods of the disclosure include those to prevent ARDS in an individual positive for COVID-19 (SARS-CoV-2). The methods may reduce the number of symptoms, reduce the severity of one or more symptoms, delay the onset of one or more symptoms, reduce the likelihood of having one or more particular symptoms (e.g., lung-related or potentially deadly symptoms), or a combination thereof.

Utilization of fibroblasts as a substitute for MSC has occurred by the inventors. In addition to studies that demonstrated efficacy of fibroblasts in differentiating into chondrocytes in vivo and generating improvement in animal models of degenerative disc disease, a group independent from the inventors reported similar findings. Strongly supporting development of fibroblast-based products as an alternative to MSC are the recent FDA clearance to initiate clinical trials using fibroblasts. Previous studies have shown that adipose [13-16], bone marrow [17-36], placental [37], amniotic membrane [38, 39], umbilical cord [40-46], menstrual blood [47], and lung [48, 49], origin, as well as conditioned media [50-57], have demonstrated reduction of pulmonary injury, water leakage, and neutrophil accumulation.

The present disclosure encompasses administration of fibroblasts into an individual at risk for, or suffering from ARDS, or positive or at risk for COVID-19. The individual may or may not be high risk for COVID-19, including being obese, having high blood pressure, having heart disease, being diabetic, a combination thereof, and so forth. Certain methods may be used as a means to increase T regulatory cell (Treg or Tregs) numbers for an individual, wherein upon administration the Tregs endow a therapeutic effect in the individual with ARDS or at risk for ARDS. Specific T-regulatory cells for use in the current disclosure can be isolated from numerous known tissues, including peripheral blood [58], adipose stromal vascular fraction [59-62], cord blood [63-67], bone marrow [68], commercially, and/or mobilized peripheral blood [69], as examples.

The disclosure also provides methods of additionally expanding Tregs subsequent to administration of fibroblasts. In one embodiment of the disclosure, allogeneic fibroblasts are administered into the individual in order to evoke differentiation to FoxP3-expressing Treg cells from naïve T cells. The FoxP3-expressing Treg cells may be specific for antigens found in the lung, or they may be antigen non-specific. The disclosure encompasses therapeutic effects of Treg cells in both antigen-specific and antigen-nonspecific means.

In one embodiment of the disclosure, Tregs generated in vivo by administration of fibroblasts (or generated in vitro upon manipulation of fibroblasts, followed by administration of the Tregs to the individual) possess an ability to prevent death of pulmonary epithelial cells. In one specific embodiment, said Treg cells protect type 2 epithelial cells from death and allow for production of surfactant. In one embodiment, Treg cells produced in any manner encompassed herein accelerate neutrophil apoptosis. In one embodiment, Treg cells produced in any manner encompassed herein promote an M2 macrophage environment in order to reduce pulmonary inflammation. In one embodiment, Treg cells produced in any manner encompassed herein suppress cytokine storm. Irrespective of mechanisms, and not being bound by theory, the disclosure provides means of modifying the pulmonary environment by Treg expansion/activation in order to prevent, ameliorate, delay the onset of, reduce the severity of, or reverse ARDS.

In some embodiments, the disclosure encompasses use of fibroblast-derived products, such as exosomes derived from fibroblasts, to induce generation of Treg cells, either ex vivo or in vivo. Previous studies have shown that exosomes from mesenchymal stem cells possess Treg augmenting activity. Methodologies from those studies can be applied to fibroblast exosomes [70-75].

In some embodiments, fibroblasts are used to generate Treg cells in vitro or ex vivo, which are subsequently administered to an individual in vivo. In some embodiments, the Treg cells are administered to an individual simultaneously or sequentially with fibroblasts and/or fibroblast-derived products. The clinical use of T-regulatory cells has been previously described by numerous investigators and the means of expansion of T-regulatory cells can be applied to the current disclosure. Examples of previous studies include: Safinia et al. reported the manufacture of clinical grade Tregs from prospective liver transplant recipients via a CliniMACS-based GMP isolation technique and expanded using anti-CD3/CD28 beads, IL-2 and rapamycin. They showed the enrichment of a pure, stable population of Tregs (>95% CD4(+)CD25(+)FOXP3(+)), reaching adequate numbers for their clinical application. The protocol proved successful in, influencing the expansion of superior functional Tregs, as compared to freshly isolated cells, whilst also preventing their conversion to Th17 cells under pro-inflammatory conditions [76]. The disclosure encompasses addition of fibroblasts to cytokine and antibody cocktails which induce Treg generation that can enhance quantity and quality of Tregs useful for treatment of ARDS. Numerous Treg generation/expansion protocols are disclosed in the art and incorporated by reference [77-79].

Additionally, various means of expanding Treg in vivo may also be added to protocols utilizing fibroblast administration in order to enhance in vivo Treg generation [80-83]. In one embodiment, administration of Intravenous Immunoglobulin (IVIG) is disclosed as a means alone, or in combination with fibroblasts to treat ARDS. Concentrations of IVIG and clinical protocols are disclosed in the literature [84-87].

The T-regulatory cells may be expanded using protocols known in the art. In some embodiments, methods for expanding T-regulatory cells are used. In some embodiments, the method comprises culturing T-regulatory cells with particular compositions, optionally for a certain duration of time. In particular cases, T-regulatory cells are cultured for at least 1, 2, 3, 4, 5, 6, 7, or more days. In addition, or alternatively, the T-regulatory cells are cultured under a certain level of oxygen and/or in the presence of certain cytokine(s) and/or in the presence of certain antibodies. In specific cases, the culture has the following conditions: 0.5-5% oxygen, 5-100 U/ml of IL-2, and/or the presence of anti-CD3 and anti-CD28 antibodies. In some embodiments TGF-beta may be added optionally.

In some embodiments, the T-regulatory cells to be expanded are CD4 positive and/or CD25 positive. In a plurality of T-regulatory cells, in some embodiments at least 90, 91, 92, 93, 94, 95, 96, 97, 98, or 99% of the cells are CD4 positive and CD25 positive T-regulatory cells and FoxP3 positive. In some embodiments, the T-regulatory cells comprise human cells. In some embodiments, the T-regulatory cells are cultured under 1% oxygen, such as no more than or at least 0.1, 0.2, 0.3, 0.4, 0.5, 0.6, 0.7, 0.8, or 0.9% oxygen.

In some embodiments, T-regulatory cells are isolated (e.g., from a culture medium) after culturing. In some embodiments, T-regulatory cells are isolated after at least 1, 2, 3, 4, 5, 6, 7, 8, 9, or 10 or more days of culture. Additionally or alternatively, in some embodiments T-regulatory cells expressing increased CTLA-4 and/or increased IL-10 levels as compared to control T-regulatory cells are isolated (e.g., from the culture medium, and/or from cells not expressing increased CTLA-4 and/or IL-10 levels) and utilized therapeutically. In some embodiments, the T-regulatory cells are contacted with one or more agents that increase intracellular cyclic AMP (cAMP) levels and are utilized therapeutically.

In particular embodiments, T-regulatory cells, within the context of the present disclosure, encompass cells expressing a Foxp3 protein. Foxp3 is expressed by CD4+CD25+ Tregs, and gain-of-function, overexpression and analysis of Foxp3-deficient Scurfy (sf) mice show Foxp3 is at least useful to the development and maintenance of murine Tregs. All naturally occurring murine CD4+CD25+ Treg cells express Foxp3. TGF-beta1 can convert naive CD4+CD25− T cells to CD4+CD25+ Tregs via induction of Foxp3. Unlike CTLA-4, GITR and CD25, murine Foxp3 mRNA expression appears stable irrespective of T cell activation. Various surface proteins (CTLA-4, GITR, LAG-3, neuropilin-1) and cytokines (TGF-beta, IL-10) are expressed by Tregs, and like sf mice, mice lacking TGF-beta, CTLA-4 or CD25 die from autoimmunity. Human X-linked neonatal diabetes mellitus, enteropathy and endocrinopathy (IPEX) syndrome results in most cases from mutations in the forkhead/winged-helix domain of FOXP3 that disrupt critical DNA interactions; in sf mice, a frameshift mutation results in a protein lacking the forkhead domain. More than 20 mutations of FOXP3 are reported in IPEX syndrome, and the syndrome is lethal if untreated. By contrast, overexpression of murine Foxp3 gene leads to hypocellular peripheral lymphoid tissues with fewer T cells and a hypoactive immune state. Hence, control of Foxp3 levels within a certain range is useful for optimal ability to suppress ARDS. In some embodiments, the disclosure teaches assessment of FoxP3 in Treg cells and using this as a biomarker to adjust dosage of fibroblasts during therapy.

In particular embodiments, T-regulatory cells are isolated and enriched for CD4 positive, CD25 positive and optionally CD127 positive cells. By way of example, but not by way of limitation, in some embodiments, human peripheral blood mononuclear cells are separated from peripheral blood by density centrifugation using Ficoll. In some embodiments, peripheral blood mononuclear cells are labeled with anti-CD4, anti-CD25 and anti-CD127 antibodies and CD4 positive, CD25 med-hi, CD127 low cells are isolated as Treg by, e.g., FACS Aria II Cell Sorter. In some embodiments, cells are further enriched for FoxP3. In some embodiments, about 85%, 86%, 87%, 88%, 89%, 90%, 91%, 92%, 93%, 94%, 95%, 96%, 97%, 98%, 99% of the isolated and enriched Treg cells are CD4 positive and CD25 positive. In some embodiments, about 93% or greater, e.g., about 94%, 95%, 96%, 97%, 98%, 99% of the isolated and enriched Treg cells are CD4 positive and CD25 positive. In some embodiments, about 85%, 86%, 87%, 88%, 89%, 90%, 91%, 92%, 93%, 94%, 95%, 96%, 97%, 98%, 99% of the isolated and enriched CD4 positive, CD25 positive cells are FoxP3 positive. In some embodiments, about 95% of the isolated and enriched CD4 positive, CD25 positive cells are FoxP3 positive.

In some embodiments of the disclosure, individuals are administered fibroblasts have been gene modified, such as for enhanced angiogenic cytokine production to enhance efficacy of Treg cell therapy. Genes with angiogenic ability include: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, Ill, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, EG-VEGF, or a combination thereof.

In one embodiment of the disclosure, anti-CD3 antibody is given 14 days before administration of fibroblast and/or Treg cells In one specific embodiment, a 14-day course of the anti-CD3 monoclonal antibody utilizes the antibody hOKT3γ1(Ala-Ala) administered intravenously (1.42 μg per kilogram of body weight on day 1; 5.67 μg per kilogram on day 2; 11.3 μg per kilogram on day 3; 22.6 μg per kilogram on day 4; and 45.4 μg per kilogram on days 5 through 14); these doses were based on those previously used for treatment of transplant rejection [88] which is incorporated by reference. Other types of anti-CD3 molecules and dosing regimens may be used in the context of ARDS therapeutics, and the doses may be chosen from examples of utility of anti-CD3 from the literature, as described in the following papers and incorporated by reference: prevention of kidney [89-97], liver [98-100], pancreas [101-103], lung [104], and heart [105-109] transplant rejection; prevention of graft versus host disease [110], multiple sclerosis [111], type 1 diabetes [112],

The use of monoclonal antibodies for the practice of the disclosure is tempered by the caution that in some cases cytokine storm may be initiated by antibody administration [113, 114]. In some cases, this is concentration-dependent [115]. Treatment for this can be accomplished by steroid administration and/or anti-IL6 antibody [116-120].

In some embodiments of the disclosure, administration of PGE1 and/or various natural anti-inflammatory compounds are provided to decrease TNF-alpha production as a result of anti-CD3 administration, such as was described and which is incorporated by reference herein [121]. In further embodiments of the disclosure, administration of anti-CD3 may be performed together with one or more endothelial protectants and/or one or more anti-coagulants in order to reduce clotting associated with CD3 modulating agents [122]. In some embodiments, anti-CD3 antibodies may be used in combination with tolerogenic cytokines, such as interleukin-10, in order to enhance number of angiogenesis supporting T cells. The safety of anti-CD3 and IL-10 administration has previously been demonstrated in a clinical trial [123].

In the current disclosure, decreased TNF-alpha activity is correlated with enhancement of pulmonary regenerative activity. Furthermore, other inhibitors of TNF-alpha may be administered [124, 125]. Etanercept, infliximab, certolizumab, golimumab, and adalimumab are examples of TNF-alpha inhibitors that may be used.

In some embodiments of the disclosure, enhancement of pulmonary regenerative activity is provided by administration of oral modulators of CD3. Oral administration of OKT3 has been previously performed in a clinical trial and results are incorporated by reference [126, 127].

In some embodiments of the disclosure, fibroblast-derived products, such as exosomes, are administered to enhance generation of Treg cells in vivo. Exosomes, also referred to as “particles,” may comprise vesicles or a flattened sphere limited by a lipid bilayer. The particles may comprise diameters of 40-100 nm. The particles may be formed by inward budding of the endosomal membrane. The particles may have a density of about 1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in fibroblasts or conditioned medium. The particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes for which the fibroblasts are utilized.

In one embodiment, fibroblast-derived products, such as exosomes, or particles may be produced by culturing fibroblast cells in a medium to condition it. The fibroblast cells may comprise human umbilical tissue derived cells (or from other sources) that possess markers selected from the group consisting of CD90, CD73, CD105, and a combination thereof. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or beta-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. It may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₕ of particles in this peak is about 45-55 nm. Such fractions comprise mesenchymal stem cell particles such as exosomes.

Culture-conditioned media may be concentrated by filtering/desalting means known in the art. In one embodiment, Amicon filters, or substantially equivalent means, with specific molecular weight cut-offs are utilized, and the cut-offs may select for molecular weights higher than 1 kDa to 50 kDa.

The cell culture supernatant may alternatively be concentrated using means known in the art, such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4° C.

Before testing the eluate for activity in vitro, the methanol is evaporated under nitrogen and replaced by culture medium. C18 cartridges are used to adsorb small hydrophobic molecules from the stem or progenitor cell culture supernatant, and allows for the elimination of salts and other polar contaminants. It may, however be desired to use other adsorption means in order to purify certain compounds from said fibroblast cell supernatant. Said fibroblast concentrated supernatant may be assessed directly for biological activities useful for the practice of this disclosure, or may be further purified. In one embodiment, said supernatant of fibroblast culture is assessed for ability to stimulate proteoglycan synthesis using an in vitro bioassay. Said in vitro bioassay allows for quantification and knowledge of which molecular weight fraction of supernatant possesses biological activity. Bioassays for testing ability to stimulate proteoglycan synthesis are known in the art. Production of various proteoglycans can be assessed by analysis of protein content using techniques including mass spectrometry, column chromatography, immune based assays such as enzyme linked immunosorbent assay (ELISA), immunohistochemistry, and flow cytometry.

Further purification may be performed using, for example, gel filtration using a Bio-Gel P-2 column with a nominal exclusion limit of 1800 Da (Bio-Rad, Richmond, Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Amniotic fluid stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In some embodiments of the methods, one or more adjuvants are utilized. In any method of composition herein, the adjuvants may comprise one or more peptides, including at least peptides are selected from the group consisting of: BPC-157, beta thymosine, Pam3CysSerLys4,functional derivatives thereof, and a mixture thereof. The adjuvants may comprise one or more activators of one or more toll like receptors. For example, for toll like receptor 2, the activator of toll like receptor 2 may be selected from the group consisting of: PAM2CSK4, beta glucan, water insoluble fractions of medicinal mushrooms (Lentinula edodes, Grifola frondosa, Hypsizygus marmoreus varieties, Flammulina velutipes), Diprovocim, HSPA4, HSPA5,HSPA9, HSPA13, HSPD1,VCAN, Lipoproteins LprG and LpqH, MTB lipoprotein Rv1016c, HKLM n) FSL-1, and a mixture thereof. For toll like receptor 3, and activator of toll like receptor 3 may be selected from the group consisting of: Poly IC, ARNAX, double-stranded RNA, and a mixture thereof. For TLR-4, the activator of TLR-4 may be LPS, Buprenorphine, Carbamazepine, Fentanyl, Levorphanol, Methadone, Cocaine, Morphine, Oxcarbazepine, Oxycodone, Pethidine, Glucuronoxylomannan from Cryptococcus, Morphine-3-glucuronide, lipoteichoic acid, β-defensin 2, small molecular weight hyaluronic acid, fibronectin EDA, snapin, tenascin C, or a mixture thereof. For TLR-5, the activator of TLR-5 may be flagellin. For TLR-6, the activator may be FSL-1. For TLR-7, the activator of TLR-7 may be imiquimod. For TLR-8, the activator of TLR8 may be ssRNA40/LyoVec. For TLR-9, the activator of TLR-9 may be a CpG oligonucleotide, ODN2006, and/or Agatolimod.

In some embodiments, the adjuvant is chloroquine and/or hydroxychloroquine or functionally active derivatives thereof. The hydroxychloroquine may be administered at a concentration and frequency sufficient to reduce viral replication. The hydroxychloroquine may be administered at a concentration and frequency sufficient to reduce activation of a TLR, such as TLR-9. The hydroxychloroquine may be administered at a concentration and frequency sufficient to protect pulmonary type 2 epithelial cells. In specific embodiments, the hydroxychloroquine is administered at a concentration and frequency sufficient to reduce production of one or more inflammatory cytokines in the lung. The inflammatory cytokine may be selected from the group consisting of: interleukin-1, interleukin-6, interleukin-8, interleukin-11, interleukin-15, interleukin-17, interleukin-18, interleukin-23, TNF-alpha, angiopoietin, HMGB-1, and a combination thereof. In some cases, the adjuvant is resveratrol, losartan, and/or azithromycin, or functionally active derivatives thereof.

In some embodiments, one or more peptides are utilized as adjuvants in conjunction with fibroblast therapy. In some cases, the peptide comprises BPC-157 (GEPPPGKPADDAGLV; SEQ ID NO:1) or a functional derivative thereof. The peptide may comprise, consist of, or consist essentially of SEQ ID NO:1. In some cases, the peptide comprises SEQ ID NO:1 but also comprises an N-terminal extension and/or a C-terminal extension. The peptide comprising SEQ ID NO:1 may be 16, 17, 18, 19, 20, 25, 30, 35, 40, 45, 50, or more amino acids in length. In some cases the peptide comprises a sequence having 1, 2, 3, 4, or 5 or more amino acid substitutions compared to SEQ ID NO:1, and the substitutions may or may not be conservative. A functional derivative of SEQ ID NO:1 may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1.

In some cases, the peptide comprises beta thymosine (SDKPDMAEI EKFDKSKLKK TETQEKNPLP SKETIEQEKQ AGES; SEQ ID NO:2) or a functional derivative thereof. The peptide may comprise, consist of, or consist essentially of SEQ ID NO:2. In some cases, the peptide comprises SEQ ID NO:2 but also comprises an N-terminal extension and/or a C-terminal extension. The peptide comprising SEQ ID NO:1 may be 44, 45, 46, 47, 48, 49, 50, 51, 52, 53, 54, 55, 56, 57, 58, 59, 60, 61, 62, 63, 64, 65, or more amino acids in length. In some cases the peptide comprises a sequence having 1, 2, 3, 4, 5, 6, 7, 8, 9, 10, or more amino acid substitutions compared to SEQ ID NO:2, and the substitutions may or may not be conservative. A functional derivative of SEQ ID NO:2 may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:2.

In some cases, the adjuvant comprises the synthetic bacterial lipopeptide: Pam3CysSerLys4 or a functional derivative thereof. In some cases, the peptide portion of the molecule (CysSerLys4) comprises 1, 2, or more substitutions, whether conservative or not. The peptide may also be extended in length an additional 1, 2, 3, 4, 5, 6, 7, 8, 9, 10, or more amino acids, for example.

In some embodiments, fibroblasts, together with therapeutic adjuvant(s), are administered to prevent ARDS, which is a life-threatening lung injury that allows fluid to leak into the lungs. Breathing becomes difficult and oxygen cannot get into the body. Most people who get ARDS are already at the hospital for trauma or illness. The lung injury causing ARDS is characterized by injury to the lung epithelium that leads to impaired resolution of pulmonary edema and also facilitates accumulation of protein-rich edema fluid and inflammatory cells in the distal airspaces of the lung Inflammatory mediators produced by neutrophils and macrophages as well as viruses, cause damage to the tight junctions between alveolar epithelial cells allowing pathologic flow of proteinaceous fluid into alveoli. Normal alveolar fluid clearance from the alveoli to the interstitium adequately removes any fluid accumulation, but the rate of fluid clearance is impaired by infection, inflammatory cytokines and the mechanical ventilation frequently employed in ARDS. Cytokine storm decreases the number of α-epithelial sodium channel (α-ENaC) subunits in the apical membrane of alveolar epithelial cells, which contributes to increased accumulation and impaired clearance of fluid from the alveoli. Exposure of cultured type II alveolar epithelial cells to IL-1β, TNF-α, and IFN-γ increases the protein permeability of alveoli by 5-fold over 24 hours. Impaired pulmonary function due to pulmonary capillary endothelial and alveolar epithelial cell dysfunction is exacerbated by damage to type II alveolar cells (pulmonary progenitor cells), which also interferes with normal surfactant function. Damage to the delicate alveolar-capillary barrier causes fluid accumulation in the air spaces of the lungs, significantly interfering with gas exchange in the alveoli and the clearance of the fluid. Normally, pulmonary capillary endothelial cells form a tight barrier that separates the pulmonary capillaries and the alveoli, which prevents the passage of proteinaceous fluid and inflammatory cells between these cells. Adherens junctions, formed by the association of VE-cadherin proteins on the membrane of adjacent endothelial cells, create the alveolar-capillary barrier Inflammatory cytokines and other signaling proteins present in cytokine storm disrupt these adherens junctions between endothelial cells allowing leakage of the capillaries. Neutrophils, activated platelets and bacterial products, such as endotoxin, can also damage or destroy the endothelial cells themselves, further increasing the permeability of this barrier. In some embodiments of the disclosure, the combination of fibroblasts and adjuvants is used to decrease inflammation in the lung, so methods of decreasing lung inflammation are encompassed herein. In other embodiments, the combination of fibroblasts and adjuvants is used to induce an increase in surfactant production, so methods of increasing surfactant production are encompassed herein. In other embodiments, the disclosure provides means of suppressing the loss of alveoli through protecting the type 2 pulmonary epithelial cells that are the target of coronaviruses, so methods of suppressing the loss of alveoli, such as through protecting type 2 pulmonary epithelial cells that are the target of coronavirus, are encompassed herein.

Disclosed are means of inducing a tolerogenic state in the lung of an individual susceptible to, or, suffering from acute respiratory distress syndrome (ARDS). Said tolerogenic state reduces inflammation and pathology of ARDS. In some embodiments, the disclosure teaches that administration of fibroblasts together with immature antigen presenting cells, such as monocytes and/or dendritic cells, of autologous and/or allogeneic origin, provides an environment conducive to stimulation of cells which inhibit inflammation and stimulate regeneration of damaged pulmonary cells. In one embodiment of the disclosure, patients are identified as having risk of ARDS based on typical clinical parameters and/or cytokine alterations.

In some embodiments, fibroblasts may be derived from a one or more tissues through means known in the art. Activation of NF-kappa B in fibroblasts may enhance the ability of fibroblasts to synergize with immature dendritic cells and/or monocytes and reduce ARDS pathology.

Various sources of fibroblasts may be used for the practice of the disclosure, these include: foreskin, adipose tissue, skin biopsy, bone marrow, placenta, umbilical cord, amneotic fluid, umbilical cord blood, ear lobe skin, embryonic fibroblasts, plastic surgery related by-product, nail matrix, or a combination thereof.

In some embodiments, fibroblasts are activated prior to therapeutic use. In some embodiments, fibroblasts are contacted with agents that act as “regenerative adjuvants” for said fibroblasts. The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells may express proteins characteristic of normal fibroblasts including the fibroblast-specific marker, CD90 (Thy-1), a 35 kDa cell-surface glycoprotein, and the extracellular matrix protein, collagen. In particular embodiments, the fibroblast dosage formulation comprises an autologous cell therapy product composed of a suspension of autologous fibroblasts, grown from a biopsy of an individual's own skin using standard tissue culture procedures. In some embodiments, the fibroblasts can also be used to create other cell types for tissue repair or regeneration.

The fibroblasts utilized in methods encompassed herein may be generated by outgrowth from a biopsy of the recipient's own skin (in the case of autologous preparations, for example), or skin of healthy donors (for allogeneic preparations, for example). In some embodiments fibroblasts are used from young donors. In particular embodiments, fibroblasts are transfected with genes to allow for enhanced growth and overcoming of the Hayflick limit, subsequent to derivation of cells expansion in culture using standard cell culture techniques. Skin tissue (dermis and epidermis layers) may be biopsied from a subject's post-auricular area. In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area.

Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany).

After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37±2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed.

Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology may be evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures may be evaluated. Keratinocytes appear round and irregularly shaped and, at higher confluence, they appear organized in a cobblestone formation. At lower confluence, keratinocytes are observable in small colonies.

Cells may be incubated at 37±2.0° C. with 5.0±1.0% CO₂ and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is ˜95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers.

Cells are then incubated at 37±2.0° C. with 5.0±1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In particular embodiments, along with immature dendritic cells and/or immature monocytes, about 50 million to 500 million fibroblast cells are administered to the subject. For example, about 50 million to about 100 million fibroblast cells, about 50 million to about 200 million fibroblast cells, about 50 million to about 300 million fibroblast cells, about 50 million to about 400 million fibroblast cells, about 100 million to about 200 million fibroblast cells, about 100 million to about 300 million fibroblast cells, about 100 million to about 400 million fibroblast cells, about 100 million to about 500 million fibroblast cells, about 200 million to about 300 million fibroblast cells, about 200 million to about 400 million fibroblast cells, about 200 million to about 500 million fibroblast cells, about 300 million to about 400 million fibroblast cells, about 300 million to about 500 million fibroblast cells, about 400 million to about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells or about 500 million fibroblast cells may be administered to the subject.

In some embodiments, fibroblast-derived products, such as exosomes, are used as an adjuvant to immature dendritic cells and/or monocytes. Exosomes for use in the current disclosure may be purified as follows: In one embodiment, fibroblasts are cultured using means known in the art for preserving viability and proliferative ability of fibroblasts. The disclosure may be applied both for individualized autologous exosome preparations and for exosome preparations obtained from established cell lines, for experimental or biological use. In one embodiment, this disclosure is more specifically based on the use of chromatography separation methods for preparing membrane vesicles, particularly to separate the membrane vesicles from potential biological contaminants, wherein said microvesicles are exosomes, and cells utilized for generating said exosomes are fibroblast cells.

Indeed, the applicant has now demonstrated that membrane vesicles, particularly exosomes, could be purified, and may possess ability to inhibit pain. In one embodiment, a strong or weak anion exchange may be performed. In addition, in a specific embodiment, the chromatography is performed under pressure. Thus, more specifically, it may consist of high performance liquid chromatography (HPLC). Different types of supports may be used to perform the anion exchange chromatography. These may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS®, SEPHAROSE®, SEPHADEX®, TRISACRYL®, TSK-GEL SW OR PW®, SUPERDEX® TOYOPEARL HW and SEPHACRYL®, for example, which are suitable for the application of the methods encompassed herein. Therefore, in a specific embodiment, this disclosure relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, supports may be in bead form. These beads may have a homogeneous and calibrated diameter, with a sufficiently high porosity to enable the penetration of the objects under chromatography (i.e. the exosomes). In this way, given the diameter of exosomes (generally between 50 and 100 nm), some embodiments use high porosity gels between 10 nm and 5 μm, or between approximately 20 nm and approximately 2 μm, or between about 100 nm and about 1 μm. For the anion exchange chromatography, the support used must be functionalized using a group capable of interacting with an anionic molecule. Generally, this group is composed of an amine which may be ternary or quaternary, which defines a weak or strong anion exchanger, respectively. Within the scope of this disclosure, may be advantageous to use a strong anion exchanger. In this way, according to the disclosure, a chromatography support as described above, functionalized with quaternary amines, is used. Therefore, according to a specific embodiment of the disclosure, the anion exchange chromatography is performed on a support functionalized with a quaternary amine. In some embodiments, this support should be selected from poly(styrene-divinylbenzene), acrylamide, agarose, dextran and silica, alone or in mixtures, and functionalized with a quaternary amine. Examples of supports functionalized with a quaternary amine include the gels SOURCEQ. MONO Q, Q SEPHAROSE®, POROS® HQ and POROS® QE, FRACTOGEL® TMAE type gels and TOYOPEARL SUPER® Q gels.

In some embodiments, a support to perform the anion exchange chromatography comprises poly(styrene-divinylbenzene). An example of this type of gel which may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μL up to 10 mL or greater. In this way, the supports available have a capacity which may reach 25 mg of proteins/ml, for example. For this reason, a 100 μL column has a capacity of approximately 2.5 mg of proteins which, given the samples in question, allows the treatment of culture supernatants of approximately 2 L (which, after concentration by a factor of 10 to 20, for example, represent volumes of 100 to 200 ml per preparation). It is understood that higher volumes may also be treated, by increasing the volume of the column, for example. In addition, to perform this disclosure, it is also possible to combine the anion exchange chromatography step with a gel permeation chromatography step. In this way, according to a specific embodiment of the disclosure, a gel permeation chromatography step is added to the anion exchange step, either before or after the anion exchange chromatography step. In this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX® 200HR (Pharmacia), TSK G6000 (TosoHaas) or SEPHACRYL® S (Pharmacia) may be used. The process according to the disclosure may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the disclosure, the biological sample is a culture supernatant of membrane vesicle-producing fibroblast cells.

In addition, according to some embodiments, the biological sample is treated, prior to the chromatography step, to be enriched with membrane vesicles (enrichment stage). In this way, in a specific embodiment, this disclosure relates to a method of preparing membrane vesicles from a biological sample, characterised in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a method of preparing membrane vesicles according to this disclosure more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In specific embodiments, the enrichment step comprises an affinity chromatography step, optionally preceded by a step of elimination of cells and/or cell debris (clarification). An enrichment step according to this disclosure comprises (i) the elimination of cells and/or cell debris (clarification), (ii) a concentration and (iii) an affinity chromatography. The cells and/or cell debris may be eliminated by centrifugation of the sample, for example, at a low speed, such as below 1000 g, including between 100 and 700 g, for example. Centrifugation conditions during this step may be approximately between 300 g and 600 g for a period between 1 and 15 minutes, for example.

The cells and/or cell debris may also be eliminated by filtration of the sample, possibly combined with the centrifugation described above. The filtration may be performed with successive filtrations using filters with a decreasing porosity. For this purpose, filters with a porosity above 0.2 μm, e.g. between 0.2 and 10 μm, may be used. It is particularly possible to use a succession of filters with a porosity of 10 μm, 1 μm, 0.5 μm followed by 0.22 μm.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to an embodiment, the biological sample (e.g., the supernatant) is subjected to an ultrafiltration, and in some instances a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the disclosure, the use of membranes with a cut-off threshold below 1000 kDa, or between 300 kDa and 1000 kDa, or between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. An affinity chromatography on a dye may be used, allowing the elimination (i.e., the retention) of contaminants such as proteins and enzymes, for instance albumin, kinases, deshydrogenases, clotting factors, interferons, lipoproteins, or also co-factors, etc. In some embodiments, the support used for this chromatography step is a support as used for the ion exchange chromatography, functionalised with a dye. As specific example, the dye may be selected from Blue SEPHAROSE® (Pharmacia), YELLOW 86, GREEN 5 and BROWN 10 (Sigma). The support may be agarose. It should be understood that any other support and/or dye or reactive group allowing the retention (binding) of contaminants from the treated biological sample can be used in the instant disclosure.

In one embodiment, a membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a particular embodiment, step b) above comprises a filtration of the culture supernatant, followed by an ultrafiltration, which may be tangential. In specific embodiments, step b) above comprises a clarification of the culture supernatant, followed by an affinity chromatography on dye, such as on Blue SEPHAROSE®

In addition, after step c), the material harvested may, if applicable, be subjected to one or more additional treatment and/or filtration stages d), particularly for sterilisation purposes. For this filtration treatment stage, filters with a diameter less than or equal to 0.3 μm may be used, or filters less than or equal to 0.25 μm may be used. Such filters have a diameter of 0.22 μm, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the disclosure comprises at least the following steps: c) an anion exchange chromatography and/or gel permeation chromatography treatment of the biological sample, and d) a filtration step, particularly sterilising filtration, of the material harvested after stage c). In a first variant, the process according to the disclosure comprises: c) an anion exchange chromatography treatment of the biological sample, and d) a filtration step, particularly sterilising filtration, on the material harvested after step c).

In another variant, the process according to the disclosure comprises: c) a gel permeation chromatography treatment of the biological sample, and d) a filtration step, particularly sterilising filtration, on the material harvested after step c). According to a third variant, the process according to the disclosure comprises: c) an anionic exchange treatment of the biological sample followed or preceded by gel permeation chromatography, and d) a filtration step, particularly sterilising filtration, on the material harvested after step c).

The disclosure, in some embodiments, teaches the application of immunological tolerance to the condition of ARDS. It is known that a cardinal feature of the immune system, is allowing for recognition and elimination of pathological threats, while selectively ignoring antigens that belong to the body. Traditionally, autoimmune conditions or conditions associated with cytokine storm, such as ARDS are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. The same holds true for cytokine storm conditions such as sepsis, where overproduction of agents such as TNF-alpha result in vascular leakage, coagulopathy, and death. The disclosure provides the utilization of tolerance-induction in ARDS alone, or in combination with existing techniques. The utilization of antigen-nonspecific immature dendritic cells in ARDS allows for induction of a inhibitory immune response, which results in suppression of pulmonary inflammation.

To cure or ameliorate conditions of immune overactivation in ARDS, Embodiments of the disclosure delete/inactivate one or more T cell clones that are associated with stimulation of inflammation, as well as block innate immune elements. Such embodiments are akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [129]; the “mirror image”, T regulatory (Treg) cells are actually selected to live by encounter with self-antigens, and Treg cells that do not bind self antigens are deleted [130, 131]. In one embodiment, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [132-138]. The generation of clinical-grade dendritic cells is well known and described in the art and incorporated by reference [139-263].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [264], antigen presentation in the context of PD1-L [265], and HLA-G interacting with immune inhibitory receptors such as ILT4 [266]. In pregnancy, “tolerogenic antigen presentation” occurs only through the indirect pathway of antigen presentation [267]. Other pathways of selective tolerogenesis in pregnancy include the stimulation of Treg cells, which have been demonstrated essential for successful pregnancy [268]. In the context of cancer, depletion of tumor specific T cells, while sparing of T cells with specificities to other antigens has been demonstrated by the tumor itself or tumor associated cells [269-271]. Additionally, Treg cells have been demonstrated to actively suppress anti-tumor T cells, perhaps as a “back up” mechanism of tumor immune evasion [273-275]. At a clinical level the ability of tumors to inhibit peripheral T cell activity has been associated in numerous studies with poor prognosis [276-278]. Oral tolerance is the process by which ingested antigens induce generation of antigen-specific TGF-beta producing cells (called “Th3” by some) [279-281], as well as Treg cells [282, 283]. Ingestion of antigen, including the autoantigen collagen II [284], has been shown to induce inhibition of both T and B cell responses in a specific manner [285, 286]. It appears that induction of regulatory cells, as well as deletion/anergy of effector cells is associated with antigen presentation in a tolerogenic manner [287]. Remission of disease in animal models of RA [288], multiple sclerosis [289], and type I diabetes [290], has been reported by oral administration of autoantigens. Furthermore, clinical trials have shown signals of efficacy of oral tolerance in autoimmune diseases such as rheumatoid arthritis [291], autoimmune uveitis [292], and multiple sclerosis [293]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments, the generation of immature dendritic cells is performed by either co-culture in vitro, or administration of T regulatory cells in vivo [294].

In some embodiments of the disclosure, alpha-1 antitrypsin is administered in order to induce tolerogenic dendritic cells in order to treat ARDS. The use of this compound for stimulation of immature DC has been previously described and is incorporated by reference [295].

In some embodiments, immature dendritic cells with or without other cells of the disclosure are administered to treat capillary leak syndrome and/or ARDS. Identification of these two conditions can be made based on techniques which are known in the art, and the methods described herein can be used to reduce, inhibit or alleviate at least one symptom of the disease. Symptoms of capillary leak syndrome (SCLS) include, but are not limited to, for example, low blood pressure (hypotension), hypoalbuminemia, decrease in plasma volume (hemoconcentration), fatigue, nausea, abdominal pain, extreme thirst, increase in body weight, elevated white blood count, fluid accumulation in lower limbs, watery stool, among others. Symptoms of ARDS include, but are not limited to, for example, shortness of breath, cough, fever, fast heart rates, rapid breathing, chest pain, decreased oxygen levels, and pathological symptoms, including, for example, severe alveolar congestion, presence of hemorrhage, interstitial edema and increased alveolar wall thickness, among others.

In some embodiments, administration of immature dendritic cells is performed using other agents. Such agents may comprise inhaled nitric oxide (iNO), which is a vasodilator indicated for treatment of term and near-term neonates with hypoxic respiratory failure associated with clinical or echocardiographic evidence of pulmonary hypertension. In these patients, iNO has been shown to improve oxygenation and reduce the need for extracorporeal membrane oxygenation therapy. NO binds to and activates cytosolic guanylate cyclase, thereby increasing intracellular levels of cyclic guanosine 3′,5′-monophosphate (cGMP). This, in turn, relaxes vascular smooth muscle, leading to vasodilatation. Inhaled NO selectively dilates the pulmonary vasculature, with minimal systemic vasculature effect as a result of efficient hemoglobin scavenging. In acute lung injury (ALI) and acute respiratory distress syndrome (ARDS), increases in partial pressure of arterial oxygen (PaO₂) are believed to occur secondary to pulmonary vessel dilation in better-ventilated lung regions. As a result, pulmonary blood flow is redistributed away from lung regions with low ventilation/perfusion ratios toward regions with normal ratios. Unfortunately iNO works in few patients, therefore, in some embodiments, immature dendritic cells, or exosomes thereof, are used to increase the efficacy of iNO.

In some embodiments, inflammatory cytokines, especially tumor necrosis factor alpha (TNF) and interleukin 1-beta (IL-1) are reduced by administration of immature dendritic cells. It is known that these inflammatory cytokines are major mediators that can elicit changes in cell phenotype, especially causing a variety of morphological and gene expression changes in endothelial cells. With respect to coagulation, one of the clot-promoting and one of the inhibitory pathways seem especially prone to modulation by these cytokines. In one embodiment, administration of immature dendritic cells is performed in order to reduce potential for coagulopathy.

It is known that whenever tissue factor contacts the blood, coagulation is initiated rapidly. In one embodiment, the immature dendritic cells reduce tissue factor expression by endothelial cells, or cytokines that produce this effect. These cytokines, TNF and IL-1, can elicit tissue factor production on endothelium and monocytes. Therefore, in one embodiment, dendritic cells are administered in order to induce a profound systemic reduction of IL-1 and TNF at a concentration of modulation sufficient to prevent disseminated intravascular coagulation.

In normal physiological situations, tissue factor is located exclusively in the extravascular space, largely on fibroblasts, where it is expressed constitutively. Furthermore, cytokines, especially interleukin 6 (IL-6), can stimulate new platelet formation, and the new platelets responding to IL-6 have increased sensitivity to thrombin activation and increased procoagulant activity. Regulating the clotting process are a large number of anticoagulant and fibrinolytic mechanisms. The three major anticoagulant mechanisms appear to involve antithrombin-heparin, tissue factor pathway inhibitor (TFPI) and the Protein C pathway. Of these, the Protein C pathway appears to be the primary target for cytokine action. The Protein C pathway is initiated when thrombin binds to thrombomodulin (TM).

In one embodiment, fibroblasts are utilized to induce upregulation of anti-coagulative proteins. TM is expressed constitutively on endothelium. In tissue culture, TNF, IL-1 or endotoxin lead to a slow loss of TM and endothelial cell Protein C receptor (EPCR) from the cell surface. In addition, Protein S levels decrease in patients with disseminated intravascular coagulation (DIC). Taken together, these results suggest that cytokines should elicit massive thrombotic responses when administered systemically. At near toxic levels, TNF fails to elicit an overt DIC or thrombotic response in patients, although sensitive markers of coagulation do detect changes in coagulation in response to TNF. In one embodiment, concentrations of TNF and IL-1, as well as pro-coagulant pathway components and anti-coagulant components are used to guide concentration of immature dendritic cell administration. In baboons, very high levels of TNF also fail to elicit fibrinogen or platelet consumption. However, if the Protein C pathway is blocked, these cytokines can elicit either DIC or deep-vein thrombosis, depending on the conditions. Thrombus formation is potently potentiated by impeding flow and/or by catheterization. DIC is facilitated by providing membrane surfaces, possibly mimicking complement mediated platelet activation/damage that occurs in shock [296]. In one embodiment, microvesicles, such as exosomes for example, produced by immature dendritic cells are used to modulate the thrombogenicity of the blood vessel surface to inhibit DIC.

In one embodiment of the disclosure, immature dendritic cells are utilized to allow for augmentation of endothelial anti-thrombotic functions after a patient receives paclitaxel. In one specific embodiment paclitaxel is given to an ARDS patient and immature dendritic cells are administered to reduce potential thrombosis. Studies have shown that tissue factor pathway inhibitor expression was reduced by prolonged treatment with either paclitaxel or TNF-alpha [297]. In one embodiment, immature dendritic cells are administered to increase expression of tissue factor pathway inhibitor expression.

In specific embodiments, immature dendritic cells are utilized as biological regulator of inflammation. Under normal conditions, inflammation is a protective response by an organism to fend off an invading agent Inflammation is a cascading event that involves many cellular and humoral mediators. On one hand, suppression of inflammatory responses can leave a host immunocompromised, however, if left unchecked, inflammation can lead to serious complications including chronic inflammatory diseases (e.g. asthma, psoriasis, arthritis, rheumatoid arthritis, multiple sclerosis, inflammatory bowel disease and the like), septic shock and multiple organ failure. Importantly, these diverse disease states share common inflammatory mediators, such as cytokines, chemokines, inflammatory cells and other mediators secreted by these cells. Immature dendritic cells may be utilized to inhibit pathological inflammation while allowing various aspects of the immune response to remain intact.

Generally, inflammatory conditions, infection-associated conditions or immune-mediated inflammatory disorders may be prevented or treated by administration of the immature dendritic cells with or without other cells encompassed herein. Examples of such inflammatory conditions include sepsis-associated conditions, inflammatory bowel diseases, autoimmune disorders, inflammatory disorders and infection-associated conditions. It is also thought that cancers, cardiovascular and metabolic conditions, neurologic and fibrotic conditions can be prevented or treated by administration of the TLR3 antibody antagonists of the disclosure. Inflammation may affect a tissue or be systemic. Exemplary affected tissues are the respiratory tract, lung, the gastrointestinal tract, small intestine, large intestine, colon, rectum, the cardiovascular system, cardiac tissue, blood vessels, joint, bone and synovial tissue, cartilage, epithelium, endothelium, hepatic or adipose tissue. Exemplary systemic inflammatory conditions are cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, severe viral infections, influenza, pneumonia, shock, or sepsis.

Inflammatory conditions that are treatable with immature dendritic cells include sepsis-associated condition that may include systemic inflammatory response syndrome (SIRS), septic shock or multiple organ dysfunction syndrome (MODS). dsRNA released by viral, bacterial, fungal, or parasitic infection and by necrotic cells can contribute to the onset of sepsis. While not wishing to be bound by an particular theory, it is believed that treatment with immature dendritic cells can provide a therapeutic benefit by extending survival times in patients suffering from sepsis-associated inflammatory conditions or prevent a local inflammatory event (e.g., in the lung) from spreading to become a systemic condition, by potentiating innate antimicrobial activity, by demonstrating synergistic activity when combined with antimicrobial agents, by minimizing the local inflammatory state contributing to the pathology, or any combination of the foregoing. Such intervention may be sufficient to permit additional treatment (e.g., treatment of underlying infection or reduction of cytokine levels) necessary to ensure patient survival. Sepsis can be modeled in animals, such as mice, by the administration of D-galactosamine and poly(I:C). In such models, D-galactosamine is a hepatotoxin which functions as a sepsis sensitizer and poly(I:C) is a sepsis-inducing molecule that mimics dsRNA and activates TLR3. immature dendritic cells treatment may increase animal survival rates in a murine model of sepsis, and thus ppMSC may be useful in the treatment of sepsis.

Some embodiments encompass the treatment of gastrointestinal inflammation by immature dendritic cells with or without other cells encompassed herein. Specifically, gastrointestinal inflammation is inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Mucosal layer may be mucosa of the bowel (including the small intestine and large intestine), rectum, stomach (gastric) lining, or oral cavity.

Exemplary chronic gastrointestinal inflammatory conditions include inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), infections colitis, ischemic colitis, collagenous or lymphocytic colitis, necrotizing enterocolitis, colitis in conditions such as chronic granulomatous disease or celiac disease, food allergies, gastritis, infectious gastritis or enterocolitis (e.g., Helicobacter pylori-infected chronic active gastritis) and other forms of gastrointestinal inflammation caused by an infectious agent Inflammatory bowel disease (IBD) includes a group of chronic inflammatory disorders of generally unknown etiology, e.g., ulcerative colitis (UC) and Crohn's disease (CD). Clinical and experimental evidence suggest that the pathogenesis of IBD is multifactorial involving susceptibility genes and environmental factors. In inflammatory bowel disease, the tissue damage results from an inappropriate or exaggerated immune response to antigens of the gut microflora. Several animal models for inflammatory bowel diseases exist. Some of the most widely used models are the 2,4,6-trinitrobenesulfonic acid/ethanol (TNBS)-induced colitis model or the oxazalone model, which induce chronic inflammation and ulceration in the colon. Another model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. Another model involves the adoptive transfer of naive CD45RB.sup.high CD4 T cells to RAG or SCID mice. In this model, donor naive T cells attack the recipient gut causing chronic bowel inflammation and symptoms similar to human inflammatory bowel diseases.

The administration of immature dendritic cells encompassed in any embodiment herein can be used to evaluate the potential efficacy of those antagonists to ameliorate symptoms and alter the course of diseases associated with inflammation in the gut, such as inflammatory bowel disease. Several treatment options for IBD are available, for example anti-TNF-.alpha. antibody therapies have been used for a decade to treat Crohn's disease. However, a significant percentage of patients are refractory to the current treatments, and thus immature dendritic cells with or without other cells encompassed herein are promising compositions in the treatment of these conditions. In some embodiments, the use of immature dendritic cells together with anti-TNF alpha antibodies are envisioned.

In some embodiments, immature dendritic cells, with or without other cells encompassed herein, are utilized to treat COVID-19 induced, or other types of induced Systemic Inflammatory Response Syndrome (SIRS). According to the accepted definition, this is a term characterizing an inflammatory syndrome caused by infectious or traumatic causes in which patients exhibit at least 2 of the following criteria: 1) body temperature less than 36° C. or greater than 38° C.; 2) heart rate greater than 90 beats per minute; 3) tachypnea, with greater than 20 breaths per minute; or, an arterial partial pressure of carbon dioxide less than 4.3 kPa (32 mmHg: 4) white blood cell count less than 4000 cells/mm³ (4×109 cells/L) or greater than 12,000 cells/mm³ (12×109 cells/L); or the presence of greater than 10% immature neutrophils (band forms) [298]. SIRS is different than sepsis in that in sepsis an active infection is found [299]. These patients may progress to acute kidney or lung failure, shock, and multiple organ dysfunction syndrome. The term septic shock refers to conditions in which the patient has a systolic blood pressure of less than 90 mmHg despite sufficient fluid resuscitation and administration of vasopressors/inotropes.

It is to be noted that immature dendritic cells may be generated with the concept of addressing major issues associated with SIRS. Predominant events in the progression to SIRS and subsequently to multiorgan failure (MOR) include: systemic activation of inflammatory responses [300], endothelial activation and initiation of the clotting cascade, associated with consumption of anticoagulants and fibrinolytic factors [301], complement activation [302], and organ failure and death.

These pathological events appear to be related to each other. For example, it is known that complement activation stimulates the pro-coagulant state [303]. In the cancer patient, SIRS may be initiated by several factors. Numerous patients receive immune suppressive chemo/radiotherapies that promote opportunistic infections [304, 305]. Additionally, given that approximately 40-70% of patients are cachectic, the low grade inflammation causing the cachexia could augment effects of additional bacterial/injury-induced inflammatory cascades [306]. Finally, tumors themselves, and through interaction with host factors, have been demonstrated to generate systemically-acting inflammatory mediators such as IL-1, IL-6, and TNF-alpha that may predispose to SIRS [307, 308].

Current SIRS treatments are primarily supportive. To date, the only drug to have elicited an effect on SIRS in Phase III double-blind, placebo-controlled trials has been Xigris (activated protein C (APC)) [309], which exerts its effects by activating endothelial cell-protecting mechanisms mediating protection against apoptosis, stimulation of barrier function through the angiopoietin/Tie-2 axis, and by reducing local clotting [310-312]. The basis of approval for Xigris has been questioned by some [313] and, additionally, it is often counter-indicated in oncology-associated sepsis (especially leukemias where bleeding is an issue of great concern). In fact, in the Phase III trials of Xigris, hematopoietic transplant patients were excluded [314]. Thus there is a great need for progress in the area of SIRS treatment and adjuvant approaches for agents such as Xigris. In one embodiment of the disclosure, APC is administered as Xigris.

One of the main causes of death related to SIRS is dysfunction of the microcirculatory system, which in the most advanced stages is manifested as disseminated intravascular coagulation (DIC) [301]. In one embodiment, immature dendritic cells with or without other cells encompassed herein are utilized to inhibit onset of DIC. Without being bound to theory, immature dendritic cells are generated in a manner to inhibit inflammatory mediators associated with SIRS, whether endotoxin or injury-related signals such as TLR agonists or HMGB-1, are all capable of activating endothelium systemically [315, 316]. Under physiological conditions, the endothelial response to such mediators is local and provides a useful mechanism for sequestering an infection and allowing immune attack. In SIRS, the fact that the response is systemic causes disastrous consequences including organ failure. The characteristics of this endothelial response include: upregulation of tissue factor (TF) [317, 318] and suppression of endothelial inhibitors of coagulation such as protein C and the antithrombin system causing a pro-coagulant state [319], increased expression of adhesion molecules which elicit, in turn, neutrophil extravasation [320], decreased fibrinolytic capacity [321-323], and increased vascular permeability/non-responsiveness to vaso-dilators and vasoconstrictors [324, 325]. Excellent detailed reviews of molecular signals associated with SIRS-induced endothelial dysfunction have been published[326-334] and one of the key factors implicated has been NF-kB [335]. Nuclear translocation of NF-kB is associated with endothelial upregulation of pro-thrombotic molecules and suppressed fibrinolysis [336-338]. In an elegant study, Song et al. inhibited NF-kB selectively in the endothelium by creation of transgenic mice transgenic expressing exogenous i-kappa B (the NF-kB inhibitor) specifically in the vasculature. In contrast to wild-type animals, the endothelial cells of these transgenic mice experienced substantially reduced expression of tissue factor while retaining expression of endothelial protein C receptor and thrombomodulin subsequent to endotoxin challenge. Furthermore, expression of NF-B was associated with generation of TNF-alpha as a result of TACE activity [339].

It is interesting that the beneficial effects of Xigris in SIRS appear to be associated with its ability to prevent the endothelial dysfunction [340] associated with suppression of proinflammatory chemokines [341], prevention of endothelial cell apoptosis [342], and increased endothelial fibrinolytic activity [343, 344]. Some of the protective activities of Xigris have been ascribed to its ability to suppress NF-kB activation in endothelial cells [345, 346]. Another example of conditions that immature dendritic cells are useful for treatment of is an inflammatory pulmonary condition. Exemplary inflammatory pulmonary conditions include infection-induced pulmonary conditions including those associated with viral, bacterial, fungal, parasite or prion infections; allergen-induced pulmonary conditions; pollutant-induced pulmonary conditions such as asbestosis, silicosis, or berylliosis; gastric aspiration-induced pulmonary conditions, immune dysregulation, inflammatory conditions with genetic predisposition such as cystic fibrosis, and physical trauma-induced pulmonary conditions, such as ventilator injury. These inflammatory conditions also include asthma, emphysema, bronchitis, chronic obstructive pulmonary disease (COPD), sarcoidosis, histiocytosis, lymphangiomyomatosis, acute lung injury, acute respiratory distress syndrome, chronic lung disease, bronchopulmonary dysplasia, community-acquired pneumonia, nosocomial pneumonia, ventilator-associated pneumonia, sepsis, viral pneumonia, influenza infection, parainfluenza infection, rotavirus infection, human metapneumovirus infection, respiratory syncitial virus infection and Aspergillus or other fungal infections. Exemplary infection-associated inflammatory diseases may include viral or bacterial pneumonia, including severe pneumonia, cystic fibrosis, bronchitis, airway exacerbations and acute respiratory distress syndrome (ARDS). Such infection-associated conditions may involve multiple infections such as a primary viral infection and a secondary bacterial infection.

Several clinical studies have supported the possibility that ascorbic acid (AA) mediates a beneficial effect on endothelial cells, especially in the context of chronic stress. Accordingly, in one embodiment of the disclosure immature dendritic cells are utilized together with AA. Heitzer et al. [347] examined acetylcholine-evoked endothelium-dependent vaso-responsiveness in 10 chronic smokers and 10 healthy volunteers. While responsiveness was suppressed in smokers, administration of intra-arterial ascorbate was capable of augmenting reactivity: an augmentation evident only in the smokers. Endothelial stress induced in 17 healthy volunteers by administration of L-methionine led to decreased responsiveness to hyperemic flow and increased homocysteine levels. Oral AA (1 g/day) restored endothelial responsiveness [348]. Restoration of endothelial responsiveness by AA has also been reported in patients with insulin-dependent [348] and independent diabetes [350], as well as chronic hypertension [351]. In these studies AA was administered intraarterially or intravenously, and the authors proposed the mechanism of action to be increased nitric oxide (NO) as a result of AA protecting it from degradation by reactive oxygen species (ROS).

A closer look at the literature suggests that there are several general mechanisms by which AA may exert endothelial protective properties. The importance of basal production of NO in endothelial function comes from its role as a vasodilator, and an inhibitor of platelet aggregation [352, 353]. High concentrations of NO are pathological in SIRS due to induction of vascular leakage [354]. However, lack of NO is also pathological because it causes loss of microvascular circulation and endothelial responsiveness [355, 356]. Although there are exceptions, the general concept is that inducible nitric oxide synthase (iNOS) and neuronal nitric oxide synthase (nNOS) are associated with sepsis-induced pathologies, whereas eNOS is associated with protective benefits [357]. It is important to note that, while iNOS expression occurs in almost all major cells of the body in the context of inflammation, eNOS is constitutively expressed by the endothelium. AA administration decreases iNOS in the context of inflammation [358, 359], but appears to increase eNOS [360]. Thus, AA appears to increase local NO concentrations through: prevention of ROS-mediated NO inactivation [361, 362], increased activity of endothelial-specific nitric oxide synthase (eNOS) [363], possibly mediated by augmenting bioavailability of tetrahydrobiopterin [364-369], a co-factor of eNOS [370], and induction of NO release from plasma-bound S-nitrosothiols [360].

In addition to deregulation of NO, numerous other endothelial changes occur during SIRS, including endothelial cell apoptosis, upregulation of adhesion molecules, and the procoagulant state [371]. AA has been reported to be active in modulating each of these factors. Rossig et al. reported that in vitro administration of AA led to reduction of TNF-alpha induced endothelial cell apoptosis [109]. The effect was mediated in part through suppression of the mitochondria-initiated apoptotic pathway as evidenced by reduced caspase-9 activation and cytochrome c release. To extend their study into the clinical realm, the investigators prospectively randomized 34 patients with NYHA class III and IV heart failure to receive AA or placebo treatment. AA treatment (2.5 g administered intravenously and 3 days of 4 g per day oral AA) Resulted in reduction in circulating apoptotic endothelial cells in the treated but not placebo control group [372]. Various mechanisms for inhibition of endothelial cell apoptosis by AA have been proposed including upregulation of the anti-apoptotic protein bcl-2 [373] and the Rb protein, suppression of p53 [374], and increasing numbers of newly formed endothelial progenitor cells [375].

AA has been demonstrated to reduce endothelial cell expression of the adhesion molecule ICAM-1 in response to TNF-alpha in vitro in human umbilical vein endothelial (HUVEC) cells (HUVEC) [376]. By reducing adhesion molecule expression, AA suppresses systemic neutrophil extravasation during sepsis, especially in the lung [377]. Other endothelial effects of AA include suppression of tissue factor upregulation in response to inflammatory stimuli [378], and effect expected to prevent the hypercoaguable state. Furthermore, ascorbate supplementation has been directly implicated in suppressing endothelial permeability in the face of inflammatory stimuli [379-381], which would hypothetically reduce vascular leakage. Given the importance of NF-kappa B signaling in coordinating endothelial inflammatory changes [336-338], it is important to note that AA at pharmacologically attainable concentrations has been demonstrated to specifically inhibit this transcription factor on endothelial cells [382]. Mechanistically, several pathways of inhibition have been identified including reduction of i-kappa B phosphorylation and subsequent degradation [383], and suppression of activation of the upstream p38 MAPK pathway [384]. In vivo data in support of eventual use in humanshas been reported showing that administration of 1 g per day AA in hypercholesterolemic pigs results in suppression of endothelial NF-kappa B activity, as well as increased eNOS, NO, and endothelial function [385]. In another porcine study, renal stenosis was combined with a high cholesterol diet to mimic renovascular disease. AA administered i.v. resulted in suppression of NF-kappa B activation in the endothelium, an effect associated with improved vascular function [386].

An important factor in reports of clinical studies of AA is the difference in effects seen when different routes of administration are employed. Supplementation with oral AA appears to have rather minor effects, perhaps due to the rate-limiting uptake of transporters found in the gut. Indeed, maximal absorption of AA appears to be achieved with a single 200 mg dose [387]. Higher doses produce gut discomfort and diarrhea because of effects of ascorbate accumulation in the intestinal lumen [388]. This is why some studies use parenteral administration. An example of the superior biological activity of parenteral versus oral was seen in a study administering AA to sedentary men. Parenteral but not oral administration was capable of augmenting endothelial responsiveness as assessed by a flow-mediated dilation assay [389].

In some embodiments, immature dendritic cells are administered together with mesenchymal stem cells. “Mesenchymal stem cell” or “MSC” in some embodiments refers to cells that are (1) adherent to plastic, (2) express CD73, CD90, and CD105 antigens, while being CD14, CD34, CD45, and HLA-DR negative, are of autologous and/or allogeneic origin, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: regenerative activity, production of growth factors, ability to induce a healing response, either directly, or through elicitation of endogenous host repair mechanisms.

### III. KITS

Any of the compositions described herein may be comprised in a kit. In a non-limiting example, the kit comprises fibroblasts, immune regulatory cells, and/or one or more adjuvants for fibroblasts. The immune regulatory cells may be of any kind, including T cells, B cells, or mixtures thereof. The kit may comprise any kind of adjuvants, including particular peptides (BPC-157; beta thymosine, and Pam₃CysSerLys₄), activators of toll like receptors, hydroxychloroquine, resveratrol, losartan, azithromycin, and so forth.

The kits may comprise a suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the compositions and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly considered. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### IV. EXAMPLES

The following example is included to demonstrate particular embodiments of the disclosure. It should be appreciated by those of skill in the art that the techniques disclosed in the example that follows represent techniques discovered by the inventor to function well in the practice of the embodiments of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Improvement of Lung Edema

C57/BL6 female mice (10 per group) were intraperitoneally injected with 50 mg/kg pentobarbital. Lipopolysaccharides (LPS) (5 mg/kg) (Sigma-Aldrich) was delivered to the lungs through a tracheostomy. Umbilical cord blood mononuclear cells were selected for expression of CD25 using Magnetic Activated Cell Sorting (MACS). Cells (500,000 cells in 150 μL PBS) were administered via the tail vein 6 h after LPS administration. Some animals received fibroblasts at the same concentration, or a combination of fibroblasts and Treg cells.

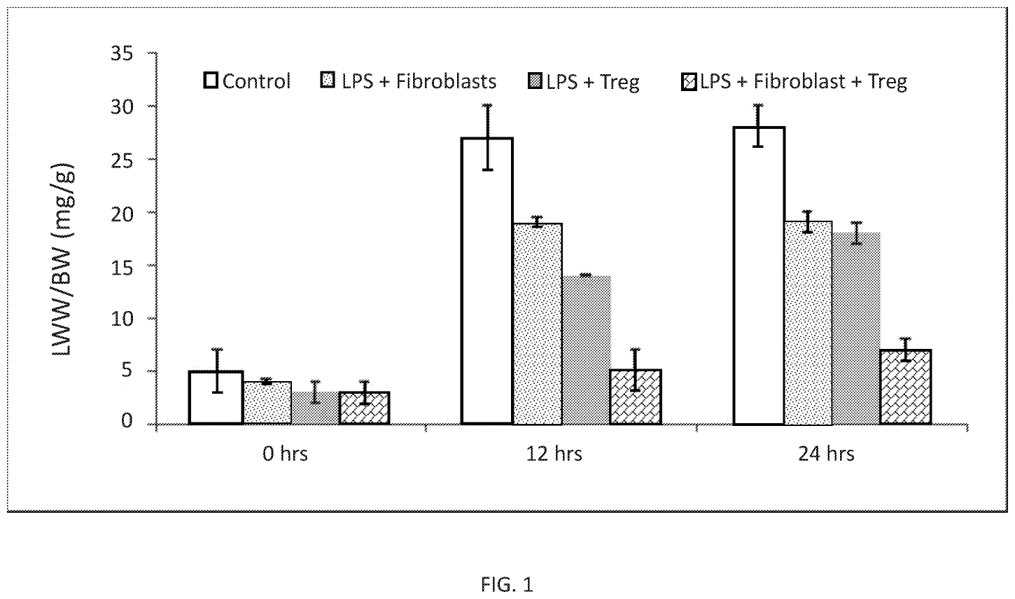

Animals were sacrificed at 0 hrs, 12, and 24 hrs. Lung edema was assessed by quantify the ratio of lung wet weight to body weight ratios (LWW/BW) (FIG. 1).

### REFERENCES

All patents and publications mentioned in the specification are indicative of the level of those skilled in the art to which the disclosure pertains. All patents and publications are herein incorporated by reference herein to the same extent as if each individual publication was specifically and individually indicated to be incorporated by reference.

1. WHO, W.H.O., Coronavirus disease (COVID-19) outbreak 2020: p. https://www.who.int/emergencies/diseases/novel-coronavirus-2019.

2. Zhang, S., et al., Estimation of the reproductive number of Novel Coronavirus (COVID-19) and the probable outbreak size on the Diamond Princess cruise ship: A data-driven analysis. Int J Infect Dis, 2020.

3. Zhao, S., et al., Preliminary estimation of the basic reproduction number of novel coronavirus (2019-nCoV) in China, from 2019 to 2020: A data-driven analysis in the early phase of the outbreak. Int J Infect Dis, 2020. 92: p. 214-217.

4. Huang, C., et al., Clinical features of patients infected with 2019 novel coronavirus in Wuhan, China. Lancet, 2020. 395(10223): p. 497-506.

5. Wang, D., et al., Clinical Characteristics of 138 Hospitalized Patients With 2019 Novel Coronavirus-Infected Pneumonia in Wuhan, China. JAMA, 2020.

6. Chen, N., et al., Epidemiological and clinical characteristics of 99 cases of 2019 novel coronavirus pneumonia in Wuhan, China: a descriptive study. Lancet, 2020. 395(10223): p. 507-513.

7. Grasselli, G., A. Pesenti, and M. Cecconi, Critical Care Utilization for the COVID-19 Outbreak in Lombardy, Italy: Early Experience and Forecast During an Emergency Response. JAMA, 2020.

8. Wu, C., et al., Risk Factors Associated With Acute Respiratory Distress Syndrome and Death in Patients With Coronavirus Disease 2019 Pneumonia in Wuhan, China. JAMA Intern Med, 2020.

9. Shi, H., et al., Radiological findings from 81 patients with COVID-19 pneumonia in Wuhan, China: a descriptive study. Lancet Infect Dis, 2020.

10. Xu, Z., et al., Pathological findings of COVID-19 associated with acute respiratory distress syndrome. Lancet Respir Med, 2020.

11. Tian, S., et al., Pulmonary pathology of early phase 2019 novel coronavirus (COVID-19) pneumonia in two patients with lung cancer. J Thorac Oncol, 2020.

12. Guo, Y. R., et al., The origin, transmission and clinical therapies on coronavirus disease 2019 (COVID-19) outbreak—an update on the status. Mil Med Res, 2020. 7(1): p. 11.

13. Jung, Y. J., et al., The effect of human adipose-derived stem cells on lipopolysaccharide-induced acute respiratory distress syndrome in mice. Ann Transl Med, 2019. 7(22): p. 674.

14. Chen, C. H., et al., Effective protection against acute respiratory distress syndrome/sepsis injury by combined adipose-derived mesenchymal stem cells and preactivated disaggregated platelets. Oncotarget, 2017. 8(47): p. 82415-82429.

15. Lu, H., et al., Pulmonary Retention of Adipose Stromal Cells Following Intravenous Delivery Is Markedly Altered in the Presence of ARDS. Cell Transplant, 2016. 25(9): p. 1635-1643.

16. Zheng, G., et al., Treatment of acute respiratory distress syndrome with allogeneic adipose-derived mesenchymal stem cells: a randomized, placebo-controlled pilot study. Respir Res, 2014. 15: p. 39.

17. Lu, Z., et al., Mesenchymal stem cells induce dendritic cell immune tolerance via paracrine hepatocyte growth factor to alleviate acute lung injury. Stem Cell Res Ther, 2019. 10(1): p. 372.

18. Silva, J. D., et al., Mesenchymal Stromal Cells Are More Effective Than Their Extracellular Vesicles at Reducing Lung Injury Regardless of Acute Respiratory Distress Syndrome Etiology. Stem Cells Int, 2019. 2019: p. 8262849.

19. Xu, A. L., et al., Mesenchymal Stem Cells Reconditioned in Their Own Serum Exhibit Augmented Therapeutic Properties in the Setting of Acute Respiratory Distress Syndrome. Stem Cells Transl Med, 2019. 8(10): p. 1092-1106.

20. Cardenes, N., et al., Cell therapy for ARDS: efficacy of endobronchial versus intravenous administration and biodistribution of MAPCs in a large animal model. BMJ Open Respir Res, 2019. 6(1): p. e000308.

21. Li, L., et al., Mesenchymal stem cells with downregulated Hippo signaling attenuate lung injury in mice with lipopolysaccharideinduced acute respiratory distress syndrome. Int J Mol Med, 2019. 43(3): p. 1241-1252.

22. Mokhber Dezfouli, M. R., et al., Intrapulmonary autologous transplant of bone marrow-derived mesenchymal stromal cells improves lipopolysaccharide-induced acute respiratory distress syndrome in rabbit. Crit Care, 2018. 22(1): p. 353.

23. Schwede, M., et al., Effects of bone marrow-derived mesenchymal stromal cells on gene expression in human alveolar type II cells exposed to TNF-alpha, IL-1 beta, and IFN-gamma. Physiol Rep, 2018. 6(16): p. e13831.

24. Masterson, C., et al., Syndecan-2-positive, Bone Marrow-derived Human Mesenchymal Stromal Cells Attenuate Bacterial-induced Acute Lung Injury and Enhance Resolution of Ventilator-induced Lung Injury in Rats. Anesthesiology, 2018. 129(3): p. 502-516.

25. Park, J., et al., Expression profile of microRNAs following bone marrow-derived mesenchymal stem cell treatment in lipopolysaccharide-induced acute lung injury. Exp Ther Med, 2018. 15(6): p. 5495-5502.

26. Pedrazza, L., et al., Mesenchymal stem cells improves survival in LPS-induced acute lung injury acting through inhibition of NETs formation. J Cell Physiol, 2017. 232(12): p. 3552-3564.

27. Yang, Y., et al., The Vascular Endothelial Growth Factors-Expressing Character of Mesenchymal Stem Cells Plays a Positive Role in Treatment of Acute Lung Injury In vivo. Mediators Inflamm, 2016. 2016: p. 2347938.

28. Moodley, Y., et al., Human mesenchymal stem cells attenuate early damage in a ventilated pig model of acute lung injury. Stem Cell Res, 2016. 17(1): p. 25-31.

29. Hayes, M., et al., Mesenchymal stromal cells are more effective than the MSC secretome in diminishing injury and enhancing recovery following ventilator-induced lung injury. Intensive Care Med Exp, 2015. 3(1): p. 29.

30. Monsel, A., et al., Therapeutic Effects of Human Mesenchymal Stem Cell-derived Microvesicles in Severe Pneumonia in Mice. Am J Respir Crit Care Med, 2015. 192(3): p. 324-36.

31. Hao, Q., et al., Study of Bone Marrow and Embryonic Stem Cell-Derived Human Mesenchymal Stem Cells for Treatment of Escherichia coli Endotoxin-Induced Acute Lung Injury in Mice. Stem Cells Transl Med, 2015. 4(7): p. 832-40.

32. Devaney, J., et al., Human mesenchymal stromal cells decrease the severity of acute lung injury induced by E. coli in the rat. Thorax, 2015. 70(7): p. 625-35.

33. Asmussen, S., et al., Human mesenchymal stem cells reduce the severity of acute lung injury in a sheep model of bacterial pneumonia. Thorax, 2014. 69(9): p. 819-25.

34. Shalaby, S. M., et al., Mesenchymal stromal cell injection protects against oxidative stress in Escherichia coli-induced acute lung injury in mice. Cytotherapy, 2014. 16(6): p. 764-75.

35. Bustos, M. L., et al., Activation of human mesenchymal stem cells impacts their therapeutic abilities in lung injury by increasing interleukin (IL)-10 and IL-1RN levels. Stem Cells Transl Med, 2013. 2(11): p. 884-95.

36. Rojas, M., et al., Infusion of freshly isolated autologous bone marrow derived mononuclear cells prevents endotoxin-induced lung injury in an ex-vivo perfused swine model. Stem Cell Res Ther, 2013. 4(2): p. 26.

37. Yan, X., et al., Nrf2/Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.

38. Cui, P., et al., Human amnion-derived mesenchymal stem cells alleviate lung injury induced by white smoke inhalation in rats. Stem Cell Res Ther, 2018. 9(1): p. 101.

39. Zhang, S., et al., Nrf2 transfection enhances the efficacy of human amniotic mesenchymal stem cells to repair lung injury induced by lipopolysaccharide. J Cell Biochem, 2018. 119(2): p. 1627-1636.

40. Huang, Z., et al., Transcriptomic analysis of lung tissues after hUC-MSCs and FTY720 treatment of lipopolysaccharide-induced acute lung injury in mouse models. Int Immunopharmacol, 2018. 63: p. 26-34.

41. Xuan, Y. Y., et al., Human Mesenchymal Stem/Stromal Cells From Human Umbilical Cord Ameliorate Acute Respiratory Distress Syndrome in Rats: Factors to Consider. Crit Care Med, 2017. 45(7): p. e736-e737.

42. Lee, F. Y., et al., Xenogeneic human umbilical cord-derived mesenchymal stem cells reduce mortality in rats with acute respiratory distress syndrome complicated by sepsis. Oncotarget, 2017. 8(28): p. 45626-45642.

43. Zhu, H., et al., Therapeutic Effects of Human Umbilical Cord-Derived Mesenchymal Stem Cells in Acute Lung Injury Mice. Sci Rep, 2017. 7: p. 39889.

44. Curley, G. F., et al., Cryopreserved, Xeno-Free Human Umbilical Cord Mesenchymal Stromal Cells Reduce Lung Injury Severity and Bacterial Burden in Rodent Escherichia coli-Induced Acute Respiratory Distress Syndrome. Crit Care Med, 2017. 45(2): p. e202-e212.

45. Chang, Y., et al., Intratracheal administration of umbilical cord blood-derived mesenchymal stem cells in a patient with acute respiratory distress syndrome. J Korean Med Sci, 2014. 29(3): p. 438-40.

46. Moodley, Y., et al., Human umbilical cord mesenchymal stem cells reduce fibrosis of bleomycin-induced lung injury. Am J Pathol, 2009. 175(1): p. 303-13.

47. Xiang, B., et al., Transplantation of Menstrual Blood-Derived Mesenchymal Stem Cells Promotes the Repair of LPS-Induced Acute Lung Injury. Int J Mol Sci, 2017. 18(4).

48. Wang, L., et al., Lung-Resident Mesenchymal Stem Cells Promote Repair of LPS-Induced Acute Lung Injury via Regulating the Balance of Regulatory T cells and Th17 cells. Inflammation, 2019. 42(1): p. 199-210.

49. Silva, J. D., et al., Mesenchymal Stem Cells From Bone Marrow, Adipose Tissue, and Lung Tissue Differentially Mitigate Lung and Distal Organ Damage in Experimental Acute Respiratory Distress Syndrome. Crit Care Med, 2018. 46(2): p. e132-e140.

50. Su, V. Y., et al., Mesenchymal Stem Cell-Conditioned Medium Induces Neutrophil Apoptosis Associated with Inhibition of the NF-kappaB Pathway in Endotoxin-Induced Acute Lung Injury. Int J Mol Sci, 2019. 20(9).

51. Mohammadipoor, A., et al., Therapeutic potential of products derived from mesenchymal stem/stromal cells in pulmonary disease. Respir Res, 2018. 19(1): p. 218.

52. Lee, J. H., J. Park, and J. W. Lee, Therapeutic use of mesenchymal stem cell-derived extracellular vesicles in acute lung injury. Transfusion, 2019. 59(S1): p. 876-883.

53. Abreu, S. C., D. J. Weiss, and P. R. Rocco, Extracellular vesicles derived from mesenchymal stromal cells: a therapeutic option in respiratory diseases? Stem Cell Res Ther, 2016. 7(1): p. 53.

54. Monsel, A., et al., Mesenchymal stem cell derived secretome and extracellular vesicles for acute lung injury and other inflammatory lung diseases. Expert Opin Biol Ther, 2016. 16(7): p. 859-71.

55. Liu, F. B., Q. Lin, and Z. W. Liu, A study on the role of apoptotic human umbilical cord mesenchymal stem cells in bleomycin-induced acute lung injury in rat models. Eur Rev Med Pharmacol Sci, 2016. 20(5): p. 969-82.

56. Chen, J., et al., Mesenchymal Stem Cell Conditioned Medium Promotes Proliferation and Migration of Alveolar Epithelial Cells under Septic Conditions In vitro via the JNK-P38 Signaling Pathway. Cell Physiol Biochem, 2015. 37(5): p. 1830-46.

57. Ionescu, L., et al., Stem cell conditioned medium improves acute lung injury in mice: in vivo evidence for stem cell paracrine action. Am J Physiol Lung Cell Mol Physiol, 2012. 303(11): p. L967-77.

58. Ramlal, R. and G. C. Hildebrandt, Advances in the Use of Regulatory T-Cells for the Prevention and Therapy of Graft-vs.-Host Disease. Biomedicines, 2017. 5(2).

59. Ichim, T. E., et al., Autologous stromal vascular fraction cells: a tool for facilitating tolerance in rheumatic disease. Cell Immunol, 2010. 264(1): p. 7-17.

60. Kahn, S., et al., A Stat6/Pten Axis Links Regulatory T Cells with Adipose Tissue Function. Cell Metab, 2017. 26(3): p. 475-492 e7.

61. Becker, M., M. K. Levings, and C. Daniel, Adipose-tissue regulatory T cells: Critical players in adipose-immune crosstalk. Eur J Immunol, 2017.

62. Pereira, S., et al., Modulation of adipose tissue inflammation by FOXP3+ Treg cells, IL-10, and TGF-beta in metabolically healthy class III obese individuals. Nutrition, 2014. 30(7-8): p. 784-90.

63. El-Chennawi, F., et al., Comparison of the percentages of CD4+ CD25high FOXP3+, CD4+ CD25low FOXP3+, and CD4+ FOXP3+ Tregs, in the umbilical cord blood of babies born to mothers with and without preeclampsia. Am J Reprod Immunol, 2017.

64. Seay, H. R., et al., Expansion of Human Tregs from Cryopreserved Umbilical Cord Blood for GMP-Compliant Autologous Adoptive Cell Transfer Therapy. Mol Ther Methods Clin Dev, 2017. 4: p. 178-191.

65. Parmar, S. and E. J. Shpall, Treg adoptive therapy: is more better? Blood, 2016. 127(8): p. 962-3.

66. Brunstein, C. G., et al., Umbilical cord blood-derived T regulatory cells to prevent GVHD: kinetics, toxicity profile, and clinical effect. Blood, 2016. 127(8): p. 1044-51.

67. Parmar, S., et al., Ex vivo fucosylation of third party human regulatory T cells enhances anti-graft-versus-host disease potency in vivo. Blood, 2015. 125(9): p. 1502-6.

68. Zhao, X. Y., et al., Higher frequency of regulatory T cells in granulocyte colony-stimulating factor (G-CSF)-primed bone marrow grafts compared with G-CSF-primed peripheral blood grafts. J Transl Med, 2015. 13: p. 145.

69. Patel, P., et al., Clinical grade isolation of regulatory T cells from G-CSF mobilized peripheral blood improves with initial depletion of monocytes. Am J Blood Res, 2015. 5(2): p. 79-85.

70. Zhang, B., et al., Mesenchymal stem cells secrete immunologically active exosomes. Stem Cells Dev, 2014. 23(11): p. 1233-44.

71. Zhang, B., et al., Mesenchymal stromal cell exosome-enhanced regulatory T-cell production through an antigen-presenting cell-mediated pathway. Cytotherapy, 2018. 20(5): p. 687-696.

72. Fathollahi, A., et al., In vitro analysis of immunomodulatory effects of mesenchymal stem cell- and tumor cell-derived exosomes on recall antigen-specific responses. Int Immunopharmacol, 2019. 67: p. 302-310.

73. Riazifar, M., et al., Stem Cell-Derived Exosomes as Nanotherapeutics for Autoimmune and Neurodegenerative Disorders. ACS Nano, 2019. 13(6): p. 6670-6688.

74. Guo, L. Y., et al., [Regulatory Effect of Exosomes Derived from Human Umbilical Cord Mesenchymal Stem Cells on Treg and TH17 Cells]. Zhongguo Shi Yan Xue Ye Xue Za Zhi, 2019. 27(1): p. 221-226.

75. Zhuansun, Y., et al., MSCs exosomal miR-1470 promotes the differentiation of CD4(+)CD25(+)FOXP3(+) Tregs in asthmatic patients by inducing the expression of P27KIP1. Int Immunopharmacol, 2019. 77: p. 105981.

76. Safinia, N., et al., Successful expansion of functional and stable regulatory T cells for immunotherapy in liver transplantation. Oncotarget, 2016. 7(7): p. 7563-77.

77. Oo, Y. H., et al., Liver homing of clinical grade Tregs after therapeutic infusion in patients with autoimmune hepatitis. JHEP Rep, 2019. 1(4): p. 286-296.

78. Mathew, J. M., et al., A Phase I Clinical Trial with Ex vivo Expanded Recipient Regulatory T cells in Living Donor Kidney Transplants. Sci Rep, 2018. 8(1): p. 7428.

79. Mancusi, A., et al., The Effect of TNF-alpha on Regulatory T Cell Function in Graft-versus-Host Disease. Front Immunol, 2018. 9: p. 356.

80. Breuer, J., et al., VLA-2 blockade in vivo by vatelizumab induces CD4+FoxP3+ regulatory T cells. Int Immunol, 2019. 31(6): p. 407-412.

81. Dolati, S., et al., Nanocurcumin improves regulatory T-cell frequency and function in patients with multiple sclerosis. J Neuroimmunol, 2019. 327: p. 15-21.

82. Dai, Y. Y., et al., Stem cells from human exfoliated deciduous teeth correct the immune imbalance of allergic rhinitis via Treg cells in vivo and in vitro. Stem Cell Res Ther, 2019. 10(1): p. 39.

83. Zhao, T. X., et al., Low-dose interleukin-2 in patients with stable ischaemic heart disease and acute coronary syndromes (LILACS): protocol and study rationale for a randomised, double-blind, placebo-controlled, phase I/II clinical trial. BMJ Open, 2018. 8(9): p. e022452.

84. Ahmadi, M., et al., Intravenous immunoglobulin (IVIG) treatment modulates peripheral blood Th17 and regulatory T cells in recurrent miscarriage patients: Non randomized, open-label clinical trial. Immunol Lett, 2017. 192: p. 12-19.

85. Kessel, A., et al., Intravenous immunoglobulin therapy affects T regulatory cells by increasing their suppressive function. J Immunol, 2007. 179(8): p. 5571-5.

86. Tsurikisawa, N., et al., High-dose intravenous immunoglobulin treatment increases regulatory T cells in patients with eosinophilic granulomatosis with polyangiitis. J Rheumatol, 2012. 39(5): p. 1019-25.

87. Massoud, A. H., et al., Intravenous immunoglobulin attenuates airway inflammation through induction of forkhead box protein 3-positive regulatory T cells. J Allergy Clin Immunol, 2012. 129(6): p. 1656-65 e3.

88. Woodle, E. S., et al., Phase I trial of a humanized, Fc receptor nonbinding OKT3 antibody, huOKT3gamma1(Ala-Ala) in the treatment of acute renal allograft rejection. Transplantation, 1999. 68(5): p. 608-16.

89. Cosimi, A. B., et al., Treatment of acute renal allograft rejection with OKT3 monoclonal antibody. Transplantation, 1981. 32(6): p. 535-9.

90. Ortho Multicenter Transplant Study, G., A randomized clinical trial of OKT3 monoclonal antibody for acute rejection of cadaveric renal transplants. N Engl J Med, 1985. 313(6): p. 337-42.

91. Debure, A., et al., One-month prophylactic use of OKT3 in cadaver kidney transplant recipients. Transplantation, 1988. 45(3): p. 546-53.

92. Oh, H. K., et al., Two low-dose OKT3 induction regimens following renal transplantation—clinical experience at a single center. Clin Transplant, 1998. 12(4): p. 343-7.

93. Opelz, G., Efficacy of rejection prophylaxis with OKT3 in renal transplantation. Collaborative Transplant Study. Transplantation, 1995. 60(11): p. 1220-4.

94. Darby, C. R., et al., Reduced dose OKT3 prophylaxis in sensitised kidney recipients. Transpl Int, 1996. 9(6): p. 565-9.

95. Ciancio, G., et al., Human donor bone marrow cells can enhance hyporeactivity in renal transplantation using maintenance FK 506 and OKT3 induction therapy. Transplant Proc, 1996. 28(2): p. 943-4.

96. Wald, T. H., et al., Treatment of renal allograft rejection with T10B9.1A31 or OKT3: final analysis of a phase II clinical trial. Transplantation, 1997. 64(2): p. 274-81.

97. Kumar, M. S., et al., ATGAM versus OKT3 induction therapy in cadaveric kidney transplantation: patient and graft survival, CD3 subset, infection, and cost analysis. Transplant Proc, 1998. 30(4): p. 1351-2.

98. Cosimi, A. B., et al., A randomized clinical trial comparing OKT3 and steroids for treatment of hepatic allograft rejection. Transplantation, 1987. 43(1): p. 91-5.

99. Millis, J. M., et al., Randomized prospective trial of OKT3 for early prophylaxis of rejection after liver transplantation. Transplantation, 1989. 47(1): p. 82-8.

100. Whiting, J. F., et al., Use of low-dose OKT3 as induction therapy in liver transplantation. Transplantation, 1998. 65(4): p. 577-80.

101. Melzer, J. S., et al., The use of OKT3 in combined pancreas-kidney allotransplantation. Transplant Proc, 1990. 22(2): p. 634-5.

102. Sindhi, R., et al., Increased risk of pulmonary edema in diabetic patients undergoing preemptive pancreas transplantation with OKT3 induction. Transplant Proc, 1995. 27(6): p. 3016-7.

103. Stratta, R. J., et al., A prospective randomized trial of OKT3 vs ATGAM induction therapy in pancreas transplant recipients. Transplant Proc, 1996. 28(2): p. 917-8.

104. Ross, D. J., et al., Delayed development of obliterative bronchiolitis syndrome with OKT3 after unilateral lung transplantation. A plea for multicenter immunosuppressive trials. Chest, 1996. 109(4): p. 870-3.

105. van Gelder, T., et al., A randomized trial comparing safety and efficacy of OKT3 and a monoclonal anti-interleukin-2 receptor antibody (BT563) in the prevention of acute rejection after heart transplantation. Transplantation, 1996. 62(1): p. 51-5.

106. Delgado, J. F., et al., Induction treatment with monoclonal antibodies for heart transplantation. Transplant Rev (Orlando), 2011. 25(1): p. 21-6.

107. Kormos, R. L., et al., Monoclonal versus polyclonal antibody therapy for prophylaxis against rejection after heart transplantation. J Heart Transplant, 1990. 9(1): p. 1-9, discussion 9-10.

108. Rabinov, M., et al., Recipient selection algorithm for immunosuppression in cardiac transplantation: OKT3 vs triple therapy alone. Transplant Proc, 1992. 24(1): p. 167-8.

109. Chin, C., et al., Induction therapy for pediatric and adult heart transplantation: comparison between OKT3 and daclizumab. Transplantation, 2005. 80(4): p. 477-81.

110. Prentice, H. G., et al., Use of anti-T-cell monoclonal antibody OKT3 to prevent acute graft-versus-host disease in allogeneic bone-marrow transplantation for acute leukaemia. Lancet, 1982. 1(8274): p. 700-3.

111. Weinshenker, B. G., et al., An open trial of OKT3 in patients with multiple sclerosis. Neurology, 1991. 41(7): p. 1047-52.

112. Herold, K. C., et al., A single course of anti-CD3 monoclonal antibody hOKT3gamma1(Ala-Ala) results in improvement in C-peptide responses and clinical parameters for at least 2 years after onset of type 1 diabetes. Diabetes, 2005. 54(6): p. 1763-9.

113. Ferran, C., et al., Cytokine-related syndrome following injection of anti-CD3 monoclonal antibody: further evidence for transient in vivo T cell activation. Eur J Immunol, 1990. 20(3): p. 509-15.

114. Vasquez, E. M., A. J. Fabrega, and R. Pollak, OKT3-induced cytokine-release syndrome: occurrence beyond the second dose and association with rejection severity. Transplant Proc, 1995. 27(1): p. 873-4.

115. Norman, D. J., J. A. Kimball, and J. M. Barry, Cytokine-release syndrome: differences between high and low doses of OKT3. Transplant Proc, 1993. 25(2 Suppl 1): p. 35-8.

116. Goldman, M., et al., OKT3-induced cytokine release attenuation by high-dose methylprednisolone. Lancet, 1989. 2(8666): p. 802-3.

117. Fletcher, E. A. K., et al., Extracorporeal human whole blood in motion, as a tool to predict first-infusion reactions and mechanism-of-action of immunotherapeutics. Int Immunopharmacol, 2018. 54: p. 1-11.

118. Chatenoud, L., et al., In vivo cell activation following OKT3 administration. Systemic cytokine release and modulation by corticosteroids. Transplantation, 1990. 49(4): p. 697-702.

119. Chatenoud, L., et al., Corticosteroid inhibition of the OKT3-induced cytokine-related syndrome—dosage and kinetics prerequisites. Transplantation, 1991. 51(2): p. 334-8.

120. Bugelski, P. J., et al., Monoclonal antibody-induced cytokine-release syndrome. Expert Rev Clin Immunol, 2009. 5(5): p. 499-521.

121. Barel, D., et al., Enhanced tumor necrosis factor in anti-CD3 antibody stimulated diabetic NOD mice: modulation by PGE1 and dietary lipid. Autoimmunity, 1992. 13(2): p. 141-9.

122. Pradier, O., et al., Procoagulant effect of the OKT3 monoclonal antibody: involvement of tumor necrosis factor. Kidney Int, 1992. 42(5): p. 1124-9.

123. Wissing, K. M., et al., A pilot trial of recombinant human interleukin-10 in kidney transplant recipients receiving OKT3 induction therapy. Transplantation, 1997. 64(7): p. 999-1006.

124. Charpentier, B., et al., Evidence that antihuman tumor necrosis factor monoclonal antibody prevents OKT3-induced acute syndrome. Transplantation, 1992. 54(6): p. 997-1002.

125. DeVault, G. A., Jr., et al., The effects of oral pentoxifylline on the cytokine release syndrome during inductive OKT3. Transplantation, 1994. 57(4): p. 532-40.

126. Ilan, Y., et al., Oral administration of OKT3 monoclonal antibody to human subjects induces a dose-dependent immunologic effect in T cells and dendritic cells. J Clin Immunol, 2010. 30(1): p. 167-77.

127. Lalazar, G., et al., Oral Administration of OKT3 MAb to Patients with NASH, Promotes Regulatory T-cell Induction, and Alleviates Insulin Resistance: Results of a Phase Ha Blinded Placebo-Controlled Trial. J Clin Immunol, 2015. 35(4): p. 399-407

128. Thompson, B. T., R. C. Chambers, and K. D. Liu, Acute Respiratory Distress Syndrome. N Engl J Med, 2017. 377(6): p. 562-572.

129. Ramsdell, F. and B. J. Fowlkes, Clonal deletion versus clonal anergy: the role of the thymus in inducing self tolerance. Science, 1990. 248(4961): p. 1342-8.

130. Apostolou, I., et al., Origin of regulatory T cells with known specificity for antigen. Nat Immunol, 2002. 3(8): p. 756-63.

131. Aschenbrenner, K., et al., Selection of Foxp3+ regulatory T cells specific for self antigen expressed and presented by Aire+ medullary thymic epithelial cells. Nat Immunol, 2007. 8(4): p. 351-8.

132. Cong, Y., et al., Generation of antigen-specific, Foxp3-expressing CD4+ regulatory T cells by inhibition of APC proteosome function. J Immunol, 2005. 174(5): p.

133. Buckland, M., et al., Aspirin-treated human DCs up-regulate ILT-3 and induce hyporesponsiveness and regulatory activity in responder T cells. Am J Transplant, 2006. 6(9): p. 2046-59.

134. Jin, Y., et al., Induction of auto-reactive regulatory T cells by stimulation with immature autologous dendritic cells. Immunol Invest, 2007. 36(2): p. 213-32.

135. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.

136. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.

137. Zhang, X., et al., Generation of therapeutic dendritic cells and regulatory T cells for preventing allogeneic cardiac graft rejection. Clin Immunol, 2008. 127(3): p. 313-21.

138. Marguti, I., et al., Expansion of CD4+ CD25+ Foxp3+ T cells by bone marrow-derived dendritic cells. Immunology, 2009. 127(1): p. 50-61.

139. Nestle, F. O., et al., Vaccination of melanoma patients with peptide- or tumor lysate-pulsed dendritic cells. Nat Med, 1998. 4(3): p. 328-32.

140. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.

141. Wang, F., et al., Phase I trial of a MART-1 peptide vaccine with incomplete Freund's adjuvant for resected high-risk melanoma. Clin Cancer Res, 1999. 5(10): p. 2756-65.

142. Thurner, B., et al., Vaccination with mage-3A1 peptide pulsed mature, monocyte-derived dendritic cells expands specific cytotoxic T cells and induces regression of some metastases in advanced stage IV melanoma. J Exp Med, 1999. 190(11): p. 1669-78.

143. Thomas, R., et al., Immature human monocyte-derived dendritic cells migrate rapidly to draining lymph nodes after intradermal injection for melanoma immunotherapy. Melanoma Res, 1999. 9(5): p. 474-81.

144. Mackensen, A., et al., Phase I study in melanoma patients of a vaccine with peptide pulsed dendritic cells generated in vitro from CD34(+) hematopoietic progenitor cells. Int J Cancer, 2000. 86(3): p. 385-92.

145. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.

146. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.

147. Lau, R., et al., Phase I trial of intravenous peptide-pulsed dendritic cells in patients with metastatic melanoma. J Immunother, 2001. 24(1): p. 66-78.

148. Banchereau, J., et al., Immune and clinical responses in patients with metastatic melanoma to CD34(+) progenitor-derived dendritic cell vaccine. Cancer Res, 2001. 61(17): p. 6451-8.

149. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.

150. Palucka, A. K., et al., Single injection of CD34+ progenitor-derived dendritic cell vaccine can lead to induction of T-cell immunity in patients with stage IV melanoma. J Immunother, 2003. 26(5): p. 432-9.

151. Bedrosian, I., et al., Intranodal administration of peptide pulsed mature dendritic cell vaccines results in superior CD8+ T-cell function in melanoma patients. J Clin Oncol, 2003. 21(20): p. 3826-35.

152. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.

153. Hersey, P., et al., Phase I/II study of treatment with dendritic cell vaccines in patients with disseminated melanoma. Cancer Immunol Immunother, 2004. 53(2): p. 125-34.

154. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.

155. Palucka, A. K., et al., Spontaneous proliferation and type 2 cytokine secretion by CD4+T cells in patients with metastatic melanoma vaccinated with antigen pulsed dendritic cells. J Clin Immunol, 2005. 25(3): p. 288-95.

156. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.

157. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.

158. Salcedo, M., et al., Vaccination of melanoma patients using dendritic cells loaded with an allogeneic tumor cell lysate. Cancer Immunol Immunother, 2006. 55(7): p. 819-29.

159. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.

160. Escobar, A., et al., Dendritic cell immunizations alone or combined with low doses of interleukin-2 induce specific immune responses in melanoma patients. Clin Exp Immunol, 2005. 142(3): p. 555-68.

161. Tuettenberg, A., et al., Induction of strong and persistent MelanA/MART specific immune responses by adjuvant dendritic cell-based vaccination of stage II melanoma patients. Int J Cancer, 2006. 118(10): p. 2617-27.

162. Schadendorf, D., et al., Dacarbazine (DTIC) versus vaccination with autologous peptide pulsed dendritic cells (DC) in first-line treatment of patients with metastatic melanoma: a randomized phase III trial of the DC study group of the DeCOG. Ann Oncol, 2006. 17(4): p. 563-70.

163. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.

164. Nakai, N., et al., Vaccination of Japanese patients with advanced melanoma with peptide, tumor lysate or both peptide and tumor lysate pulsed mature, monocyte-derived dendritic cells. J Dermatol, 2006. 33(7): p. 462-72.

165. Palucka, A. K., et al., Dendritic cells loaded with killed allogeneic melanoma cells can induce objective clinical responses and MART-1 specific CD8+ T-cell immunity. J Immunother, 2006. 29(5): p. 545-57.

166. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.

167. Guo, J., et al., Intratumoral injection of dendritic cells in combination with local hyperthermia induces systemic antitumor effect in patients with advanced melanoma. Int J Cancer, 2007. 120(11): p. 2418-25.

168. O'Rourke, M. G., et al., Dendritic cell immunotherapy for stage IV melanoma. Melanoma Res, 2007. 17(5): p. 316-22.

169. Bercovici, N., et al., Analysis and characterization of antitumor T-cell response after administration of dendritic cells loaded with allogeneic tumor lysate to metastatic melanoma patients. J Immunother, 2008. 31(1): p. 101-12.

170. Hersey, P., et al., Phase I/II study of treatment with matured dendritic cells with or without low dose IL-2 in patients with disseminated melanoma. Cancer Immunol Immunother, 2008. 57(7): p. 1039-51.

171. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.

172. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.

173. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.

174. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.

175. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.

176. Nakai, N., et al., Immunohistological analysis of peptide-induced delayed-type hypersensitivity in advanced melanoma patients treated with melanoma antigen pulsed mature monocyte-derived dendritic cell vaccination. J Dermatol Sci, 2009. 53(1): p. 40-7.

177. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.

178. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.

179. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.

180. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.

181. Ribas, A., et al., Multicenter phase II study of matured dendritic cells pulsed with melanoma cell line lysates in patients with advanced melanoma. J Transl Med, 2010. 8: p. 89.

182. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.

183. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.

184. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.

185. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.

186. Steele, J. C., et al., Phase I/II trial of a dendritic cell vaccine transfected with DNA encoding melan A and gp100 for patients with metastatic melanoma. Gene Ther, 2011. 18(6): p. 584-93.

187. Kim, D. S., et al., Immunotherapy of malignant melanoma with tumor lysate-pulsed autologous monocyte-derived dendritic cells. Yonsei Med J, 2011. 52(6): p. 990-8.

188. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.

189. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.

190. Dannull, J., et al., Melanoma immunotherapy using mature DCs expressing the constitutive proteasome. J Clin Invest, 2013. 123(7): p. 3135-45.

191. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.

192. Stift, A., et al., Dendritic cell vaccination in medullary thyroid carcinoma. Clin Cancer Res, 2004. 10(9): p. 2944-53.

193. Kuwabara, K., et al., Results of a phase I clinical study using dendritic cell vaccinations for thyroid cancer. Thyroid, 2007. 17(1): p. 53-8.

194. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.

195. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.

196. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.

197. Yu, J. S., et al., Vaccination with tumor lysate pulsed dendritic cells elicits antigen-specific, cytotoxic T-cells in patients with malignant glioma. Cancer Res, 2004. 64(14): p. 4973-9.

198. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.

199. Yamanaka, R., et al., Clinical evaluation of dendritic cell vaccination for patients with recurrent glioma: results of a clinical phase I/II trial. Clin Cancer Res, 2005. 11(11): p. 4160-7.

200. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.

201. Walker, D. G., et al., Results of a phase I dendritic cell vaccine trial for malignant astrocytoma: potential interaction with adjuvant chemotherapy. J Clin Neurosci, 2008. 15(2): p. 114-21.

202. Leplina, O. Y., et al., Use of interferon-alpha-induced dendritic cells in the therapy of patients with malignant brain gliomas. Bull Exp Biol Med, 2007. 143(4): p. 528-34.

203. De Vleeschouwer, S., et al., Postoperative adjuvant dendritic cell-based immunotherapy in patients with relapsed glioblastoma multiforme. Clin Cancer Res, 2008. 14(10): p. 3098-104.

204. Ardon, H., et al., Adjuvant dendritic cell-based tumour vaccination for children with malignant brain tumours. Pediatr Blood Cancer, 2010. 54(4): p. 519-25.

205. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.

206. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.

207. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.

208. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.

209. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.

210. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.

211. Fong, B., et al., Monitoring of regulatory T cell frequencies and expression of CTLA-4 on T cells, before and after DC vaccination, can predict survival in GBM patients. PLoS One, 2012. 7(4): p. e32614.

212. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.

213. Phuphanich, S., et al., Phase I trial of a multi-epitope-pulsed dendritic cell vaccine for patients with newly diagnosed glioblastoma. Cancer Immunol Immunother, 2013. 62(1): p. 125-35.

214. Akiyama, Y., et al., alpha-type-1 polarized dendritic cell-based vaccination in recurrent high-grade glioma: a phase I clinical trial. BMC Cancer, 2012. 12: p. 623.

215. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.

216. Shah, A. H., et al., Dendritic cell vaccine for recurrent high-grade gliomas in pediatric and adult subjects: clinical trial protocol. Neurosurgery, 2013. 73(5): p. 863-7.

217. Reichardt, V. L., et al., Idiotype vaccination using dendritic cells after autologous peripheral blood stem cell transplantation for multiple myeloma—a feasibility study. Blood, 1999. 93(7): p. 2411-9.

218. Lim, S. H. and R. Bailey-Wood, Idiotypic protein pulsed dendritic cell vaccination in multiple myeloma. Int J Cancer, 1999. 83(2): p. 215-22.

219. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.

220. Reichardt, V. L., et al., Idiotype vaccination of multiple myeloma patients using monocyte-derived dendritic cells. Haematologica, 2003. 88(10): p. 1139-49.

221. Guardino, A. E., et al., Production of myeloid dendritic cells (DC) pulsed with tumor-specific idiotype protein for vaccination of patients with multiple myeloma. Cytotherapy, 2006. 8(3): p. 277-89.

222. Lacy, M. Q., et al., Idiotype-pulsed antigen presenting cells following autologous transplantation for multiple myeloma may be associated with prolonged survival. Am J Hematol, 2009. 84(12): p. 799-802.

223. Yi, Q., et al., Optimizing dendritic cell-based immunotherapy in multiple myeloma: intranodal injections of idiotype pulsed CD40 ligand-matured vaccines led to induction of type-1 and cytotoxic T-cell immune responses in patients. Br J Haematol, 2010. 150(5): p. 554-64.

224. Rollig, C., et al., Induction of cellular immune responses in patients with stage-I multiple myeloma after vaccination with autologous idiotype-pulsed dendritic cells. J Immunother, 2011. 34(1): p. 100-6.

225. Zahradova, L., et al., Efficacy and safety of Id-protein-loaded dendritic cell vaccine in patients with multiple myeloma phase II study results. Neoplasma, 2012. 59(4): p. 440-9.

226. Timmerman, J. M., et al., Idiotype pulsed dendritic cell vaccination for B-cell lymphoma: clinical and immune responses in 35 patients. Blood, 2002. 99(5): p. 1517-26.

227. Maier, T., et al., Vaccination of patients with cutaneous T-cell lymphoma using intranodal injection of autologous tumor-lysate-pulsed dendritic cells. Blood, 2003. 102(7): p. 2338-44.

228. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.

229. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.

230. Li, L., et al., Immunotherapy for patients with acute myeloid leukemia using autologous dendritic cells generated from leukemic blasts. Int J Oncol, 2006. 28(4): p. 855-61.

231. Roddie, H., et al., Phase I/II study of vaccination with dendritic-like leukaemia cells for the immunotherapy of acute myeloid leukaemia. Br J Haematol, 2006. 133(2): p. 152-7.

232. Litzow, M. R., et al., Testing the safety of clinical-grade mature autologous myeloid DC in a phase I clinical immunotherapy trial of CML. Cytotherapy, 2006. 8(3): p. 290-8.

233. Westermann, J., et al., Vaccination with autologous non-irradiated dendritic cells in patients with bcr/abl+ chronic myeloid leukaemia. Br J Haematol, 2007. 137(4): p. 297-306.

234. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22(5): p. 1007-17.

235. Palma, M., et al., Development of a dendritic cell-based vaccine for chronic lymphocytic leukemia. Cancer Immunol Immunother, 2008. 57(11): p. 1705-10.

236. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.

237. Iwashita, Y., et al., A phase I study of autologous dendritic cell-based immunotherapy for patients with unresectable primary liver cancer. Cancer Immunol Immunother, 2003. 52(3): p. 155-61.

238. Lee, W. C., et al., Vaccination of advanced hepatocellular carcinoma patients with tumor lysate-pulsed dendritic cells: a clinical trial. J Immunother, 2005. 28(5): p. 496-504.

239. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.

240. Palmer, D. H., et al., A phase II study of adoptive immunotherapy using dendritic cells pulsed with tumor lysate in patients with hepatocellular carcinoma. Hepatology, 2009. 49(1): p. 124-32.

241. El Ansary, M., et al., Immunotherapy by autologous dendritic cell vaccine in patients with advanced HCC. J Cancer Res Clin Oncol, 2013. 139(1): p. 39-48.

242. Tada, F., et al., Phase I/II study of immunotherapy using tumor antigen-pulsed dendritic cells in patients with hepatocellular carcinoma. Int J Oncol, 2012. 41(5): p. 1601-9.

243. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.

244. Hirschowitz, E. A., et al., Autologous dendritic cell vaccines for non-small-cell lung cancer. J Clin Oncol, 2004. 22(14): p. 2808-15.

245. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.

246. Yannelli, J. R., et al., The large scale generation of dendritic cells for the immunization of patients with non-small cell lung cancer (NSCLC). Lung Cancer, 2005. 47(3): p. 337-50.

247. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.

248. Antonia, S. J., et al., Combination of p53 cancer vaccine with chemotherapy in patients with extensive stage small cell lung cancer. Clin Cancer Res, 2006. 12(3 Pt 1): p. 878-87.

249. Perrot, I., et al., Dendritic cells infiltrating human non-small cell lung cancer are blocked at immature stage. J Immunol, 2007. 178(5): p. 2763-9.

250. Hirschowitz, E. A., et al., Immunization of NSCLC patients with antigen-pulsed immature autologous dendritic cells. Lung Cancer, 2007. 57(3): p. 365-72.

251. Baratelli, F., et al., Pre-clinical characterization of GMP grade CCL21-gene modified dendritic cells for application in a phase I trial in non-small cell lung cancer. J Transl Med, 2008. 6: p. 38.

252. Hegmans, J. P., et al., Consolidative dendritic cell-based immunotherapy elicits cytotoxicity against malignant mesothelioma. Am J Respir Crit Care Med, 2010. 181(12): p. 1383-90.

253. Um, S. J., et al., Phase I study of autologous dendritic cell tumor vaccine in patients with non-small cell lung cancer. Lung Cancer, 2010. 70(2): p. 188-94.

254. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.

255. Perroud, M. W., Jr., et al., Mature autologous dendritic cell vaccines in advanced non-small cell lung cancer: a phase I pilot study. J Exp Clin Cancer Res, 2011. 30: p. 65.

256. Skachkova, O. V., et al., Immunological markers of anti-tumor dendritic cells vaccine efficiency in patients with non-small cell lung cancer. Exp Oncol, 2013. 35(2): p. 109-13.

257. Hernando, J. J., et al., Vaccination with autologous tumour antigen pulsed dendritic cells in advanced gynaecological malignancies: clinical and immunological evaluation of a phase I trial. Cancer Immunol Immunother, 2002. 51(1): p. 45-52.

258. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.

259. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.

260. Kandalaft, L. E., et al., A Phase I vaccine trial using dendritic cells pulsed with autologous oxidized lysate for recurrent ovarian cancer. J Transl Med, 2013. 11: p. 149.

261. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.

262. Rong, Y., et al., A phase I pilot trial of MUC1-peptide-pulsed dendritic cells in the treatment of advanced pancreatic cancer. Clin Exp Med, 2012. 12(3): p. 173-80.

263. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.

264. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.

265. D'Addio, F., et al., The link between the PDL1 costimulatory pathway and Th17 in fetomaternal tolerance. J Immunol, 2011. 187(9): p. 4530-41.

266. Kuroki, K. and K. Maenaka, Immune modulation of HLA-G dimer in maternal-fetal interface. Eur J Immunol, 2007. 37(7): p. 1727-9.

267. Erlebacher, A., et al., Constraints in antigen presentation severely restrict T cell recognition of the allogeneic fetus. J Clin Invest, 2007. 117(5): p. 1399-411.

268. Chen, T., et al., Self-specific memory regulatory T cells protect embryos at implantation in mice. J Immunol, 2013. 191(5): p. 2273-81.

269. Harimoto, H., et al., Inactivation of tumor-specific CD8(+) CTLs by tumor-infiltrating tolerogenic dendritic cells. Immunol Cell Biol, 2013. 91(9): p. 545-55.

270. Ney, J. T., et al., Autochthonous liver tumors induce systemic T cell tolerance associated with T cell receptor down-modulation. Hepatology, 2009. 49(2): p. 471-81.

271. Cheung, A. F., et al., Regulated expression of a tumor-associated antigen reveals multiple levels of T-cell tolerance in a mouse model of lung cancer. Cancer Res, 2008. 68(22): p. 9459-68.

272. Bal, A., et al., Rapid tolerization of virus-activated tumor-specific CD8+ T cells in prostate tumors of TRAMP mice. Proc Natl Acad Sci USA, 2008. 105(35): p. 13003-8.

273. Jacobs, J. F., et al., Regulatory T cells in melanoma: the final hurdle towards effective immunotherapy? Lancet Oncol, 2012. 13(1): p. e32-42.

274. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.

275. Donkor, M. K., et al., T cell surveillance of oncogene-induced prostate cancer is impeded by T cell-derived TGF-beta1 cytokine. Immunity, 2011. 35(1): p. 123-34.

276. Whiteside, T. L., Down-regulation of zeta-chain expression in T cells: a biomarker of prognosis in cancer? Cancer Immunol Immunother, 2004. 53(10): p. 865-78.

277. Whiteside, T. L., Signaling defects in T lymphocytes of patients with malignancy. Cancer Immunol Immunother, 1999. 48(7): p. 346-52.

278. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.

279. Faria, A. M. and H. L. Weiner, Oral tolerance. Immunol Rev, 2005. 206: p. 232-59.

280. Weiner, H. L., Induction and mechanism of action of transforming growth factor-beta-secreting Th3 regulatory cells. Immunol Rev, 2001. 182: p. 207-14.

281. Fukaura, H., et al., Induction of circulating myelin basic protein and proteolipid protein-specific transforming growth factor-beta1-secreting Th3 T cells by oral administration of myelin in multiple sclerosis patients. J Clin Invest, 1996. 98(1): p. 70-7.

282. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.

283. Yamashita, H., et al., Overcoming food allergy through acquired tolerance conferred by transfer of Tregs in a murine model. Allergy, 2012. 67(2): p. 201-9.

284. Park, K. S., et al., Type II collagen oral tolerance; mechanism and role in collagen-induced arthritis and rheumatoid arthritis. Mod Rheumatol, 2009.

285. Womer, K. L., et al., A pilot study on the immunological effects of oral administration of donor major histocompatibility complex class II peptides in renal transplant recipients. Clin Transplant, 2008. 22(6): p. 754-9.

286. Faria, A. M. and H. L. Weiner, Oral tolerance: therapeutic implications for autoimmune diseases. Clin Dev Immunol, 2006. 13(2-4): p. 143-57.

287. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.

288. Thompson, H. S., et al., Suppression of collagen induced arthritis by oral administration of type II collagen: changes in immune and arthritic responses mediated by active peripheral suppression. Autoimmunity, 1993. 16(3): p. 189-99.

289. Song, F., et al., The thymus plays a role in oral tolerance induction in experimental autoimmune encephalomyelitis. Ann N Y Acad Sci, 2004. 1029: p. 402-4.

290. Hanninen, A. and L. C. Harrison, Mucosal tolerance to prevent type 1 diabetes: can the outcome be improved in humans? Rev Diabet Stud, 2004. 1(3): p. 113-21.

291. Wei, W., et al., A multicenter, double-blind, randomized, controlled phase III clinical trial of chicken type II collagen in rheumatoid arthritis. Arthritis Res Ther, 2009. 11(6): p. R180.

292. Thurau, S. R., et al., Molecular mimicry as a therapeutic approach for an autoimmune disease: oral treatment of uveitis patients with an MHC peptide crossreactive with autoantigen first results. Immunol Lett, 1997. 57(1-3): p. 193-201.

293. Weiner, H. L., et al., Double-blind pilot trial of oral tolerization with myelin antigens in multiple sclerosis. Science, 1993. 259(5099): p. 1321-4.

294. Onishi, Y., et al., Foxp3+ natural regulatory T cells preferentially form aggregates on dendritic cells in vitro and actively inhibit their maturation. Proc Natl Acad Sci USA, 2008. 105(29): p. 10113-8.

295. Ozeri, E., et al., alpha-1 antitrypsin promotes semimature, IL-10-producing and readily migrating tolerogenic dendritic cells. J Immunol, 2012. 189(1): p. 146-53.

296. Esmon, C. T., Possible involvement of cytokines in diffuse intravascular coagulation and thrombosis. Baillieres Best Pract Res Clin Haematol, 1999. 12(3): p. 343-59.

297. Wood, S. C., X. Tang, and B. Tesfamariam, Paclitaxel potentiates inflammatory cytokine-induced prothrombotic molecules in endothelial cells. J Cardiovasc Pharmacol, 2010. 55(3): p. 276-85.

298. American College of Chest Physicians/Society of Critical Care Medicine Consensus Conference: definitions for sepsis and organ failure and guidelines for the use of innovative therapies in sepsis. Crit Care Med, 1992. 20(6): p. 864-74.

299. http://www.youtube.com/watch?v=p2rEJC7He6g.

300. de Jong, H. K., T. van der Poll, and W. J. Wiersinga, The systemic pro-inflammatory response in sepsis. J Innate Immun. 2(5): p. 422-30.

301. Gando, S., Disseminated intravascular coagulation in trauma patients. Semin Thromb Hemost, 2001. 27(6): p. 585-92.

302. Guo, R. F. and P. A. Ward, C5a, a therapeutic target in sepsis. Recent Pat Antiinfect Drug Discov, 2006. 1(1): p. 57-65.

303. Silasi-Mansat, R., et al., Complement inhibition decreases the procoagulant response and confers organ protection in a baboon model of Escherichia coli sepsis. Blood. 116(6): p. 1002-10.

304. Person, A. K., D. P. Kontoyiannis, and B. D. Alexander, Fungal infections in transplant and oncology patients. Infect Dis Clin North Am. 24(2): p. 439-59.

305. Kiehn, T. E., Bacteremia and fungemia in the immunocompromised patient. Eur J Clin Microbiol Infect Dis, 1989. 8(9): p. 832-7.

306. Tisdale, M. J., Cancer cachexia. Curr Opin Gastroenterol. 26(2): p. 146-51.

307. Gelin, J., et al., Role of endogenous tumor necrosis factor alpha and interleukin 1 for experimental tumor growth and the development of cancer cachexia. Cancer Res, 1991. 51(1): p. 415-21.

308. Cahlin, C., et al., Experimental cancer cachexia: the role of host-derived cytokines interleukin (IL)-6, IL-12, interferon-gamma, and tumor necrosis factor alpha evaluated in gene knockout, tumor-bearing mice on C57 Bl background and eicosanoid-dependent cachexia. Cancer Res, 2000. 60(19): p. 5488-93.

309. Ely, E. W., G. R. Bernard, and J. L. Vincent, Activated protein C for severe sepsis. N Engl J Med, 2002. 347(13): p. 1035-6.

310. Dhainaut, J. F., et al., Drotrecogin alfa (activated) (recombinant human activated protein C) reduces host coagulopathy response in patients with severe sepsis. Thromb Haemost, 2003. 90(4): p. 642-53.

311. Minhas, N., et al., Activated protein C utilizes the angiopoietin/Tie2 axis to promote endothelial barrier function. FASEB J. 24(3): p. 873-81.

312. Loubele, S. T., H. M. Spronk, and H. Ten Cate, Activated protein C: a promising drug with multiple effects? Mini Rev Med Chem, 2009. 9(5): p. 620-6.

313. Poole, D., G. Bertolini, and S. Garattini, Errors in the approval process and post-marketing evaluation of drotrecogin alfa (activated) for the treatment of severe sepsis. Lancet Infect Dis, 2009. 9(1): p. 67-72.

314. Pastores, S. M., et al., Septic shock and multiple organ failure after hematopoietic stem cell transplantation: treatment with recombinant human activated protein C. Bone Marrow Transplant, 2002. 30(2): p. 131-4.

315. Cristofaro, P. and S. M. Opal, The Toll-like receptors and their role in septic shock. Expert Opin Ther Targets, 2003. 7(5): p. 603-12.

316. Treutiger, C. J., et al., High mobility group 1 B-box mediates activation of human endothelium. J Intern Med, 2003. 254(4): p. 375-85.

317. Lv, B., et al., High-mobility group box 1 protein induces tissue factor expression in vascular endothelial cells via activation of NF-kappaB and Egr-1. Thromb Haemost, 2009. 102(2): p. 352-9.

318. Wada, H., Y. Wakita, and H. Shiku, Tissue factor expression in endothelial cells in health and disease. Blood Coagul Fibrinolysis, 1995.6 Suppl 1: p. S26-31.

319. Levi, M., The coagulant response in sepsis and inflammation. Hamostaseologie.

30(1): p. 10-2, 14-6.

320. Munro, J. M., J. S. Pober, and R. S. Cotran, Recruitment of neutrophils in the local endotoxin response: association with de novo endothelial expression of endothelial leukocyte adhesion molecule-1. Lab Invest, 1991. 64(2): p. 295-9.

321. Lippi, G., L. Ippolito, and G. Cervellin, Disseminated intravascular coagulation in burn injury. Semin Thromb Hemost. 36(4): p. 429-36.

322. Lau, C. L., et al., Enhanced fibrinolysis protects against lung ischemia-reperfusion injury. J Thorac Cardiovasc Surg, 2009. 137(5): p. 1241-8.

323. Levi, M., M. Schouten, and T. van der Poll, Sepsis, coagulation, and antithrombin: old lessons and new insights. Semin Thromb Hemost, 2008. 34(8): p. 742-6.

324. Shapiro, N., et al., The association of endothelial cell signaling, severity of illness, and organ dysfunction in sepsis. Crit Care. 14(5): p. R182.

325. Druey, K. M. and P. R. Greipp, Narrative review: the systemic capillary leak syndrome. Ann Intern Med. 153(2): p. 90-8.

326. Dejana, E., F. Orsenigo, and M. G. Lampugnani, The role of adherens junctions and VE-cadherin in the control of vascular permeability. J Cell Sci, 2008. 121(Pt 13): p.

327. Azevedo, L. C., et al., Redox mechanisms of vascular cell dysfunction in sepsis. Endocr Metab Immune Disord Drug Targets, 2006. 6(2): p. 159-64.

328. Okajima, K., Prevention of endothelial cell injury by activated protein C: the molecular mechanism(s) and therapeutic implications. Curr Vasc Pharmacol, 2004. 2(2): p. 125-33.

329. Andersson, U. and K. J. Tracey, HMGB1 in sepsis. Scand J Infect Dis, 2003. 35(9): p. 577-84.

330. Strassheim, D., J. S. Park, and E. Abraham, Sepsis: current concepts in intracellular signaling. Int J Biochem Cell Biol, 2002. 34(12): p. 1527-33.

331. ten Cate, H., et al., Microvascular coagulopathy and disseminated intravascular coagulation. Crit Care Med, 2001. 29(7 Suppl): p. S95-7; discussion S97-8.

332. Hawiger, J., Innate immunity and inflammation: a transcriptional paradigm. Immunol Res, 2001. 23(2-3): p. 99-109.

333. Edgington, T. S., et al., Cellular immune and cytokine pathways resulting in tissue factor expression and relevance to septic shock. Nouv Rev Fr Hematol, 1992. 34 Suppl: p. S15-27.

334. Mukaida, N., et al., Novel insight into molecular mechanism of endotoxin shock: biochemical analysis of LPS receptor signaling in a cell-free system targeting NF-kappaB and regulation of cytokine production/action through beta2 integrin in vivo. J Leukoc Biol, 1996. 59(2): p. 145-51.

335. Liu, S. F. and A. B. Malik, NF-kappa B activation as a pathological mechanism of septic shock and inflammation. Am J Physiol Lung Cell Mol Physiol, 2006. 290(4): p. L622-L645.

336. Ulfhammer, E., et al., TNF-alpha mediated suppression of tissue type plasminogen activator expression in vascular endothelial cells is NF-kappaB- and p38 MAPK-dependent. J Thromb Haemost, 2006. 4(8): p. 1781-9.

337. Xu, H., et al., Selective blockade of endothelial NF-kappaB pathway differentially affects systemic inflammation and multiple organ dysfunction and injury in septic mice. J Pathol. 220(4): p. 490-8.

338. Ding, J., et al., A pivotal role of endothelial-specific NF-kappaB signaling in the pathogenesis of septic shock and septic vascular dysfunction. J Immunol, 2009. 183(6): p. 4031-8.

339. Song, D., et al., Activation of endothelial intrinsic NF-{kappa}B pathway impairs protein C anticoagulation mechanism and promotes coagulation in endotoxemic mice. Blood, 2009. 114(12): p. 2521-9.

340. Grinnell, B. W. and D. Joyce, Recombinant human activated protein C. a system modulator of vascular function for treatment of severe sepsis. Crit Care Med, 2001. 29(7 Suppl): p. S53-60; discussion S60-1.

341. Brueckmann, M., et al., Activated protein C inhibits the release of macrophage inflammatory protein-1-alpha from THP-1 cells and from human monocytes. Cytokine, 2004. 26(3): p. 106-13.

342. Cheng, T., et al., Activated protein C blocks p53-mediated apoptosis in ischemic human brain endothelium and is neuroprotective. Nat Med, 2003. 9(3): p. 338-42.

343. van Hinsbergh, V. W., et al., Activated protein C decreases plasminogen activator-inhibitor activity in endothelial cell-conditioned medium. Blood, 1985. 65(2): p. 444-51.

344. Sakata, Y., et al., Activated protein C stimulates the fibrinolytic activity of cultured endothelial cells and decreases antiactivator activity. Proc Natl Acad Sci USA, 1985. 82(4): p. 1121-5.

345. Joyce, D. E. and B. W. Grinnell, Recombinant human activated protein C attenuates the inflammatory response in endothelium and monocytes by modulating nuclear factor-kappaB. Crit Care Med, 2002. 30(5 Suppl): p. S288-93.

346. Brueckmann, M., et al., Drotrecogin alfa (activated) inhibits NF-kappa B activation and MIP-1-alpha release from isolated mononuclear cells of patients with severe sepsis. Inflamm Res, 2004. 53(10): p. 528-33.

347. Heitzer, T., H. Just, and T. Munzel, Antioxidant vitamin C improves endothelial dysfunction in chronic smokers. Circulation, 1996. 94(1): p. 6-9.

348. Chambers, J. C., et al., Demonstration of rapid onset vascular endothelial dysfunction after hyperhomocysteinemia: an effect reversible with vitamin C therapy. Circulation, 1999. 99(9): p. 1156-60.

349. Timimi, F. K., et al., Vitamin C improves endothelium-dependent vasodilation in patients with insulin-dependent diabetes mellitus. J Am Coll Cardiol, 1998. 31(3): p. 552-7.

350. Ting, H. H., et al., Vitamin C improves endothelium-dependent vasodilation in patients with non-insulin-dependent diabetes mellitus. J Clin Invest, 1996. 97(1): p. 22-8.

351. Solzbach, U., et al., Vitamin C improves endothelial dysfunction of epicardial coronary arteries in hypertensive patients. Circulation, 1997. 96(5): p. 1513-9.

352. Gao, Y., The multiple actions of NO. Pflugers Arch. 459(6): p. 829-39.

353. Jackson, W. F., The endothelium-derived relaxing factor. J Reconstr Microsurg, 1989. 5(3): p. 263-71.

354. De Cruz, S. J., N. J. Kenyon, and C. E. Sandrock, Bench-to-bedside review: the role of nitric oxide in sepsis. Expert Rev Respir Med, 2009. 3(5): p. 511-21.

355. Tyml, K., F. Li, and J. X. Wilson, Septic impairment of capillary blood flow requires nicotinamide adenine dinucleotide phosphate oxidase but not nitric oxide synthase and is rapidly reversed by ascorbate through an endothelial nitric oxide synthase-dependent mechanism. Crit Care Med, 2008. 36(8): p. 2355-62.

356. Naseem, K. M., The role of nitric oxide in cardiovascular diseases. Mol Aspects Med, 2005. 26(1-2): p. 33-65.

357. Parratt, J. R., Nitric oxide in sepsis and endotoxaemia. J Antimicrob Chemother, 1998. 41 Suppl A: p. 31-9.

358. Wu, F., K. Tyml, and J. X. Wilson, Ascorbate inhibits iNOS expression in endotoxin- and IFN gamma-stimulated rat skeletal muscle endothelial cells. FEBS Lett, 2002. 520(1-3): p. 122-6.

359. Wu, F., J. X. Wilson, and K. Tyml, Ascorbate inhibits iNOS expression and preserves vasoconstrictor responsiveness in skeletal muscle of septic mice. Am J Physiol Regul Integr Comp Physiol, 2003. 285(1): p. R50-6.

360. Ulker, S., P. P. McKeown, and U. Bayraktutan, Vitamins reverse endothelial dysfunction through regulation of eNOS and NAD(P)H oxidase activities. Hypertension, 2003. 41(3): p. 534-9.

361. Peluffo, G., et al., Superoxide-mediated inactivation of nitric oxide and peroxynitrite formation by tobacco smoke in vascular endothelium: studies in cultured cells and smokers. Am J Physiol Heart Circ Physiol, 2009. 296(6): p. H1781-92.

362. May, J. M., Z. C. Qu, and X. Li, Ascorbic acid blunts oxidant stress due to menadione in endothelial cells. Arch Biochem Biophys, 2003. 411(1): p. 136-44.

363. Heller, R., et al., L-Ascorbic acid potentiates nitric oxide synthesis in endothelial cells. J Biol Chem, 1999. 274(12): p. 8254-60.

364. Nakai, K., et al., Ascorbate enhances iNOS activity by increasing tetrahydrobiopterin in RAW 264.7 cells. Free Radic Biol Med, 2003. 35(8): p. 929-37.

365. d'Uscio, L. V., et al., Long-term vitamin C treatment increases vascular tetrahydrobiopterin levels and nitric oxide synthase activity. Circ Res, 2003. 92(1): p. 88-95.

366. Toth, M., Z. Kukor, and S. Valent, Chemical stabilization of tetrahydrobiopterin by L-ascorbic acid: contribution to placental endothelial nitric oxide synthase activity. Mol Hum Reprod, 2002. 8(3): p. 271-80.

367. Patel, K. B., et al., Oxidation of tetrahydrobiopterin by biological radicals and scavenging of the trihydrobiopterin radical by ascorbate. Free Radic Biol Med, 2002. 32(3): p. 203-11.

368. Stone, K. J. and B. H. Townsley, The effect of L-ascorbate on catecholamine biosynthesis. Biochem J, 1973. 131(3): p. 611-3.

369. Huang, A., et al., Ascorbic acid enhances endothelial nitric-oxide synthase activity by increasing intracellular tetrahydrobiopterin. J Biol Chem, 2000. 275(23): p. 17399-406.

370. Schmidt, T. S. and N. J. Alp, Mechanisms for the role of tetrahydrobiopterin in endothelial function and vascular disease. Clin Sci (Lond), 2007. 113(2): p. 47-63.

371. Keel, M. and O. Trentz, Pathophysiology of polytrauma. Injury, 2005. 36(6): p.

691-709.

372. Rossig, L., et al., Vitamin C inhibits endothelial cell apoptosis in congestive heart failure. Circulation, 2001. 104(18): p. 2182-7.

373. Haendeler, J., A. M. Zeiher, and S. Dimmeler, Vitamin C and E prevent lipopolysaccharide-induced apoptosis in human endothelial cells by modulation of Bcl-2 and Bax. Eur J Pharmacol, 1996. 317(2-3): p. 407-11.

374. Saeed, R. W., T. Peng, and C. N. Metz, Ascorbic acid blocks the growth inhibitory effect of tumor necrosis factor-alpha on endothelial cells. Exp Biol Med (Maywood), 2003. 228(7): p. 855-65.

375. Fiorito, C., et al., Antioxidants increase number of progenitor endothelial cells through multiple gene expression pathways. Free Radic Res, 2008. 42(8): p. 754-62.

376. Mo, S. J., et al., Modulation of TNF-alpha-induced ICAM-1 expression, NO and H202 production by alginate, allicin and ascorbic acid in human endothelial cells. Arch Pharm Res, 2003. 26(3): p. 244-51.

377. Martin, W. J., 2nd, Neutrophils kill pulmonary endothelial cells by a hydrogen-peroxide-dependent pathway. An in vitro model of neutrophil-mediated lung injury. Am Rev Respir Dis, 1984. 130(2): p. 209-13.

378. Chen, Y. H., et al., Anti-inflammatory effects of different drugs/agents with antioxidant property on endothelial expression of adhesion molecules. Cardiovasc Hematol Disord Drug Targets, 2006. 6(4): p. 279-304.

379. Bremond, A., et al., Regulation of HLA class I surface expression requires CD99 and p230/golgin-245 interaction. Blood, 2009. 113(2): p. 347-57.

380. Wilson, J. X., Mechanism of action of vitamin C in sepsis: ascorbate modulates redox signaling in endothelium. Biofactors, 2009. 35(1): p. 5-13.

381. Utoguchi, N., et al., Ascorbic acid stimulates barrier function of cultured endothelial cell monolayer. J Cell Physiol, 1995. 163(2): p. 393-9.

382. Bowie, A. and L. A. O'Neill, Vitamin C inhibits NF kappa B activation in endothelial cells. Biochem Soc Trans, 1997. 25(1): p. 131S.

383. Carcamo, J. M., et al., Vitamin C suppresses TNF alpha-induced NF kappa B activation by inhibiting I kappa B alpha phosphorylation. Biochemistry, 2002. 41(43): p. 12995-3002.

384. Bowie, A. G. and L. A. O'Neill, Vitamin C inhibits NF-kappa B activation by TNF via the activation of p38 mitogen-activated protein kinase. J Immunol, 2000. 165(12): p. 7180-8.

385. Rodriguez-Porcel, M., et al., Chronic antioxidant supplementation attenuates nuclear factor-kappa B activation and preserves endothelial function in hypercholesterolemic pigs. Cardiovasc Res, 2002. 53(4): p. 1010-8.

386. Chade, A. R., et al., Antioxidant intervention blunts renal injury in experimental renovascular disease. J Am Soc Nephrol, 2004. 15(4): p. 958-66.

387. Levine, M., et al., Vitamin C pharmacokinetics in healthy volunteers: evidence for a recommended dietary allowance. Proc Natl Acad Sci USA, 1996. 93(8): p. 3704-9.

388. Hathcock, J. N., et al., Vitamins E and C are safe across a broad range of intakes. Am J Clin Nutr, 2005. 81(4): p. 736-45.

389. Eskurza, I., et al., Effect of acute and chronic ascorbic acid on flow-mediated dilatation with sedentary and physically active human aging. J Physiol, 2004. 556(Pt 1): p. 315-24.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of preventing or treating Acute Respiratory Distress Syndrome (ARDS) in an individual, comprising administering to the individual an effective amount of:
(a) fibroblasts and/or fibroblast-derived exosomes under conditions to allow for said fibroblasts to stimulate generation of T regulatory cells in vivo; and/or
(b) immune regulatory cells, wherein said immune regulatory cells have been exposed ex vivo or in vitro under suitable conditions to fibroblasts and/or fibroblast-derived exosomes.

2. The method of claim 1, wherein the immune regulatory cells are obtained by culture of lymphocytes with fibroblasts to produce T cells and/or B cells.

3. The method of claim 1 or 2, wherein the immune regulatory cells are T cells.

4. The method of claim 1, 2 or 3, wherein the T regulatory cells generated in vivo are from T cell progenitors, naive T cells, Th1, Th2, Th3, Th9, Th17 T cells, or a mixture thereof.

5. The method of any one of claims 1-4, wherein the individual is positive for COVID-19 (SARS-CoV-2), is high risk for COVID-19, or has been exposed to SARS-CoV-2.

6. The method of any one of claims 1-5, wherein in (a) said fibroblasts are allogeneic, autologous or syngeneic with respect to the T regulatory cells.

7. The method of any one of claims 1-6, wherein in (b) said immune regulatory cells are allogeneic, autologous or syngeneic with respect to the individual.

8. The method of any one of claims 1-7, wherein said T regulatory cells express a marker selected from the group consisting of CD4, CD25, CD73, CD105, LAP, TGF-beta, CTLA-4, GITR ligand, neuropilin-1, CTLA-4, FoxP3, CD127, GARP, and n) a combination thereof.

9. The method of any one of claims 1-8, wherein said ARDS is comprised of neutrophil infiltration in to the alveolar space.

10. The method of any one of claims 1-9, wherein said ARDS is comprised of complement activation in the lung.

11. The method of any one of claims 1-10, wherein said ARDS is comprised of enhanced expression of one or more inflammatory cytokines.

12. The method of claim 11, wherein said inflammatory cytokines are selected from the group consisting of: IL-1, IL-6, IL-8, IL-11, IL-12, IL-18, IL-21, IL-17, IL-23, IL-27, IL33, TNF-alpha, HMGB-1, and a combination thereof.

13. The method of any one of claims 1-12, wherein said T regulatory cells express FoxP3.

14. The method of any one of claims 1-13, wherein said T regulatory cells comprise membrane bound TGF-beta.

15. The method of any one of claims 1-14, wherein said T regulatory cells suppress ability of T cells to proliferate in response to one or more mitogens.

16. The method of any one of claims 1-15, wherein said T regulatory cells suppress ability of immature dendritic cells to mature into differentiated dendritic cells.

17. The method of claim 16, wherein dendritic cell maturation is associated with upregulation of expression of one or more markers selected from the group consisting of: HLA-II, CD40, CD80, CD86, and a combination thereof.

18. The method of claim 16 or 17, wherein dendritic cell maturation is associated with enhanced ability to activate proliferation of allogeneic T cells.

19. The method of any one of claims 16-18, wherein dendritic cell maturation is associated with enhanced ability to induce production of interferon gamma from allogeneic T cells.

20. The method of any one of claims 1-19, wherein said fibroblasts are substitute for immature dendritic cells in order to stimulate Treg generation in vivo.

21. The method of any one of claims 1-20, wherein fibroblasts are administered with immature dendritic cells in order to stimulate Treg generation in vivo.

22. The method of any one of claims 1-21, wherein one or more NF-kappa B inhibitors are administered to the individual.

23. The method of claim 22, wherein said NF-kappa B inhibitor is selected from the group consisting of Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, and a combination thereof.

24. The method of any one of claims 1-23, further comprising administration of one or more malaria drugs.

25. The method of any one of claims 1-24, further comprising administration of one or more adjuvants.

26. The method of claim 25, wherein the one or more adjuvants comprise:
(a) one or more peptides selected from the group consisting of: BPC-157, beta thymosine, Pam₃CysSerLys₄ (SEQ ID NO: 3), functional derivatives thereof, and a mixture thereof;
(b) one or more activators of one or more toll like receptors;
(c) chloroquine, hydroxychloroquine, a functionally active derivative thereof, or a mixture thereof;
(d) resveratrol and/or a functionally active derivative thereof;
(e) losartan and/or a functionally active derivative thereof;
(f) azithromycin and/or a functionally active derivative thereof; or
(g) a combination thereof.

27. The method of claim 26, wherein the functionally active derivative of chloroquine or hydroxychloroquine is SKM13, SKM14, a metal-chloroquine, or a combination thereof.

28. The method of claim 26 or 27, wherein a functionally active derivative of resveratrol is trans-resveratrol (3,5,4′-trihydroxystilbene); cis-resveratrol; Pterostilbene (3,5-Dimethoxy-4′ Hydroxystilbene); Trimethoxystilbene; Tetramethoxystilbene; Pentamethoxystilbene; Dihydroxystilbene; Tetrahydroxystilbene; Hexahydroxystilbene; 4′-Bromoresveratrol; 3,4,5-Trimethoxy-4!-bromo-trans-stilbene (BTS); 3,4,5-Trimethoxy-4′-bromo-cis-stilbene (BCS); 2-Chlororesveratrol; 4-Iodoresveratrol; or a combination thereof.

29. The method of any one of claims 26-28, wherein the functionally active derivative of losartan is a Losartan Nitroderivative.

30. The method of any one of claims 26-29, wherein the functionally active derivative of azithromycin is 4″-O-(benzamido)alkyl carbamates of 11,12-cyclic carbonate AZM; 4″-O-(benzamido)butyl carbamates of 11,12-cyclic carbonate AZM; or combinations thereof.

31. A method for generating a T cell population capable of suppressing pulmonary edema, wherein said T cell population comprises CD4+CD25+ regulatory T cells that are generated from freshly isolated CD4+CD25− T cells, the method comprising: isolating CD4+CD25− T cells from a sample comprising T cells obtained from a mammalian individual; contacting the isolated CD4+CD25− T cells in a culture vessel with one or more CD4+CD25+ induction agents for a time period sufficient to generate CD4+CD25+ regulatory T cells; and selecting the CD4+CD25+ cells.

32. The method of claim 31, wherein said induction agent is a population of fibroblasts.

33. The method of claim 31 or 32, wherein said population of fibroblast is allogeneic to the T cell population.

34. The method of claim 31 or 32, wherein said population of fibroblast is autologous to the T cell population.

35. The method of claim 31 or 32, wherein said population of fibroblast is xenogeneic to the T cell population.

36. The method of any one of claims 31-35, wherein CD4+CD25+ T cells express FoxP3.

37. The method of claim 31, wherein said regulatory T cells are capable of in vitro cell-to-cell contact dependent suppression of the proliferation of freshly isolated CD4+CD25− responder T cells after re-exposure to a cognate antigen.

38. The method of claim 31, further comprising expanding the CD4+CD25+ antigen-specific regulatory T cell population.

39. The method of claim 31, further comprising administering a pharmaceutically acceptable composition comprising a portion of the expanded CD4+CD25+ antigen-specific regulatory T cell population to an individual in need thereof.

40. A method of treating an inflammatory condition in an individual comprising administering to the individual a therapeutically effective amount of antigen-presenting cells and a therapeutically effective amount of one or both of fibroblasts and mesenchymal stem cells (MSCs).

41. The method of claim 40, wherein the inflammatory condition comprises cytokine storm, hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, severe viral infections, influenza, pneumonia, shock, sepsis, or a combination thereof.

42. The method of claim 40 or claim 41, wherein the antigen-presenting cells comprises a monocyte and/or a dendritic cell

43. The method of any one of claims 42, wherein the dendritic cell is an immature dendritic cell.


SEQUENCE LISTING (5 sequences)

SEQ ID NO: 1 - protein, 15 aa, Artificial Sequence
  Description of Artificial Sequence Synthetic peptide
GEPPPGKPADDAGLV

SEQ ID NO: 2 - protein, 43 aa, Artificial Sequence
  Description of Artificial Sequence Synthetic polypeptide
SDKPDMAEIEKFDKSKLKKTETQEKNPLPSKETIEQEKQAGES

SEQ ID NO: 3 - protein, 6 aa, Artificial Sequence
  Description of Artificial Sequence Synthetic peptide
CSKKKK

SEQ ID NO: 4 - protein, 6 aa, Artificial Sequence
  Description of Artificial Sequence Synthetic peptide
CSKKKK

SEQ ID NO: 5 - protein, 6 aa, Artificial Sequence
  Description of Artificial Sequence Synthetic peptide
CSKKKK